(19) United States
(12) Patent Application Publication
(10) Pub. No.: US 2014/0148729 A1
(43) Pub. Date: May 29, 2014

(54) MICRO-MECHANICAL DEVICES AND METHODS FOR BRAIN TUMOR REMOVAL

(71) Applicants: Gregory P. Schmitz, Los Gatos, CA (US); Juan Diego Perea, Campbell, CA (US); Ronald Leguidleguid, Union city, CA (US); Ming-Ting Wu, Northridge, CA (US); Gregory B. Arcenio, Redwood City, CA (US)

(72) Inventors: Gregory P. Schmitz, Los Gatos, CA (US); Juan Diego Perea, Campbell, CA (US); Ronald Leguidleguid, Union city, CA (US); Ming-Ting Wu, Northridge, CA (US); Gregory B. Arcenio, Redwood City, CA (US)

(21) Appl. No.: 14/033,397

(22) Filed: Sep. 20, 2013

Related U.S. Application Data
(60) Provisional application No. 61/731,091, filed on Nov. 29, 2012.

(51) Int. Cl. A61B 17/32; A61B 18/18; A61B 19/00; A61B 10/06; A61B 17/3207

(52) U.S. Cl. CPC A61B 17/32002; A61B 10/06; A61B 17/320758; A61B 19/2203; A61B 18/18
USPC 600/564; 606/170; 600/104; 606/159; 606/130; 606/33

(57) ABSTRACT

A method for removing at least part of a brain tumor may first involve contacting a forward-facing tissue cutter disposed at the distal end of a tissue removal device with the brain tumor. The tissue removal device may include a shaft having a diameter no greater than about 10 mm, and in some embodiments the tissue cutter does not extend laterally beyond the diameter of the shaft. The method may next involve cutting tissue from the brain tumor, using the tissue cutter. The method may then involve moving the cut tissue through a channel of the shaft in a direction from the distal end of the tissue removal device toward a proximal end of the device.

### CROSS REFERENCE TO RELATED APPLICATIONS

This application claims the benefit under 35 U.S.C. 119 of U.S. Provisional Patent Application 61/731,091 filed Nov. 29, 2012 and entitled “Micro-Mechanical Devices and Methods for Brain Tumor Removal” which is herein incorporated by reference in its entirety.

This application is related to the following U.S. applications: application Ser. No. 13/535,197 filed Jun. 27, 2012; application Ser. No. 13/388,653 filed Apr. 16, 2012; application Ser. No. 13/289,994 filed Nov. 4, 2011; application Ser. No. 13/007,578 filed Jan. 14, 2011; application Ser. No. 12/491,220 filed Jun. 24, 2009; application Ser. No. 12/490,301 filed Jun. 23, 2009; application Ser. No. 12/490,295 filed Jun. 23, 2009; Provisional Application No. 61/408,558 filed Oct. 29, 2010; Provisional Application No. 61/234,989 filed Aug. 18, 2009; Provisional Application No. 61/075,007 filed Jun. 24, 2008; Provisional Application No. 61/075,006 filed Jun. 23, 2008; Provisional Application No. 61/164,864 filed Mar. 30, 2009; Provisional Application No. 61/164,883 filed Mar. 30, 2009; application Ser. No. 13/843,462 filed Mar. 15, 2013; application Ser. No. 13/659,734 filed Oct. 24, 2012; Provisional Application No. 61/731,434 filed Nov. 29, 2012; application Ser. No. 13/714,285 filed Dec. 13, 2012; Provisional Application No. 61/731,440 filed Nov. 29, 2012; Provisional Application No. 61/710,608 filed Oct. 5, 2012; application Ser. No. 13/855,627 filed Apr. 2, 2013; Provisional Application No. 61/728,443 filed Nov. 20, 2012; Provisional Application No. 61/731,091 filed Nov. 29, 2012; and application Ser. No. 13/859,520 filed Apr. 9, 2013.

### INCORPORATION BY REFERENCE

All publications and patent applications mentioned in this specification are herein incorporated by reference to the same extent as if each individual publication or patent application was specifically and individually indicated to be incorporated by reference.

### FIELD

The field of the present application pertains to medical devices. More specifically, the present application is related to micro-mechanical devices and methods for removing brain tumors.

### DESCRIPTION OF THE RELATED ART

Brain tumors are often very challenging tumors to surgically remove, due to their close proximity to vital body tissues, specifically brain tissue and blood vessels supplying the brain with blood. Additionally, some brain tumors are located deep inside the brain or may be located next to, or even wrap themselves around, vital tissues. For these reasons, many brain tumors are inoperable or have very low rates of successful surgical intervention.

One example of a brain tumor that is somewhat challenging to remove is the pituitary tumor. The pituitary tumor forms from the pituitary gland, which is located in the sella turcica, close to the middle of the head and just beyond the sphenoid sinus. Most removal procedures involve advancing one or more removal devices through the nostrils and piercing through the sphenoid sinus and into the sella turcica to remove the tumor. FIGS. 1-6 illustrate the anatomy and the typical approach to removing pituitary tumors. One of the dangers of pituitary sinus surgery is the close proximity of the internal carotid arteries to the tumor. Another issue is that, oftentimes, the tumor material may impinge on one or both of the internal carotid arteries or another nearby structure, making removal difficult. Additionally, since the procedure is performed through the nostrils, visualization and free movement of devices may be challenging.

Many other brain tumors are even more difficult, or at least as difficult, as a pituitary tumor to remove. For example, acoustic neuromas are very difficult to remove and have a very low success rate of surgery. Open brain surgeries in general are also challenging, since any damage to adjacent brain tissue may be very damaging and life altering to the patient. A number of different tools have been proposed for removing brain tumor tissue. For example, a side cutting tissue remover is described in U.S. Patent Application Publication Numbers 2009/0124975, 2009/0228030 and 2010/0191266. One potential drawback with the side cutter described in those applications is that it may be difficult to pull tissue into the small side-hole and thus might be difficult to effectively remove tissue. The side-hole might also become easily clogged with tissue. Various radiofrequency tissue ablation devices have also been described. Although some of them may be quite effective, there is a risk of damaging nearby tissues with RF radiating out from the device.

Although many different tools for performing brain surgery exist, it would still be advantageous to have improved devices and methods. Ideally, such devices and methods would allow for small amounts of tumor to be removed precisely, without damaging surrounding tissues. Ideally, the devices would not burn or cause peripheral damage to brain tissue, blood vessels and the like. At least some of these objectives will be met by the embodiments described below.

### SUMMARY OF THE DISCLOSURE

Example embodiments described herein have several features, no single one of which is indispensable or solely responsible for their desirable attributes. Without limiting the scope of the claims, some of the advantageous features of some embodiments will now be summarized.

In one aspect, a method for removing at least part of a brain tumor may first involve contacting a forward-facing tissue cutter disposed at the distal end of a tissue removal device with the brain tumor, where the tissue removal device includes a shaft having a diameter no greater than about 10 mm, and where the tissue cutter does not extend laterally beyond the diameter of the shaft. The method may further involve cutting tissue from the brain tumor, using the tissue cutter. Finally, the method may involve moving the cut tissue through a channel of the shaft in a direction from the distal end of the tissue removal device toward a proximal end of the device.

In some embodiments, the brain tumor may be a pituitary tumor, and the method may further involve, before contacting the brain tumor: forming an incision through a spenoid sinus; and advancing the distal end of the tissue cutting device through the incision. In some embodiments, the incision may be formed using the tissue cutting device. In alternative embodiments, the brain tumor may be an acoustic neuroma. In other alternative embodiments, any other brain tumor may be removed using the method.

In many embodiments, cutting the tissue comprises shredding the tissue. For example, shredding may be on a fiber-by-fiber basis, using a tissue cutter that is very small (such as a “micro-debrider”). In some embodiments, moving the tissue may involve urging the tissue into the channel with a cutting motion of the tissue cutter. Optionally, moving the cut tissue through the channel may also involve applying suction to the channel. Additionally, moving the cut tissue through the channel may also involve introducing fluid, via the tissue removal device, to an area at or near the distal end of the tissue removal device, wherein the applied suction moves at least some of the fluid proximally through the channel with the cut tissue.

In some embodiments, the tissue cutter may include at least one moveable blade and at least one stationary blade, and cutting tissue may involve rotating the at least one rotating blade past the at least one stationary blade. In alternative embodiments, the tissue cutter may include at least two interdigitated tissue cutters, and cutting tissue may involve rotating the two interdigitated cutters toward one another. In yet other alternative embodiments, the tissue cutter may include, but is not limited to, micro-shears, graspers and/or biopsy forceps.

In some embodiments, the shaft of the tissue cutting device may have a distal tip having a length of between about 1 mm and about 25 mm, and a bend between a proximal portion of the shaft and the distal tip forming an angle between the proximal portion and the distal tip of between about 1 degree and about 90 degrees.

In some embodiments, the method may also include visualizing the cutting using a visualization device such as, but not limited to, a straight endoscope, an angled endoscope, a swing prism endoscope, a side viewing endoscope, a flexible endoscope, a CMOS digital camera, an ultrasound device and/or a scanning single fiber endoscope. In some embodiments, the visualization device may be incorporated into the tissue removal device.

In some embodiments, the method may further involve measuring an amount of the removed tissue by filtering the removed tissue from a stream of irrigation fluid. Alternatively, the method may involve measuring an amount of the removed tissue by determining motor torque in the tissue removal device during engagement of the device with the tissue and using at least one of the determined motor torque, a time period of tissue removal or a loading condition to approximate the amount of the removed tissue.

In another aspect, a device for removing at least part of a brain tumor may include: a shaft having a proximal portion, a distal tip disposed at an angle relative to the proximal portion, and a channel extending from a distal end of the distal tip through at least part of the proximal portion; at least one moveable cutting member disposed at the distal end of the distal tip and including at least two interdigated blades; a handle coupled with the proximal portion of the shaft; and an actuator coupled with the handle for actuating the at least one moveable cutting member. In some embodiments, the shaft has a diameter no greater than about 10 mm, a distal tip having a length of between about 1 mm and about 25 mm, and a bend between a proximal portion of the shaft and the distal tip forming an angle between the proximal portion and the distal tip of between about 1 degree and about 90 degrees.

In some embodiments, the channel may be a tissue removal channel extending from the distal end of the distal tip to a proximal aperture on the proximal portion through which tissue can be removed from the device. Some embodiments further include a suction port on the proximal portion or the handle for applying suction to the channel. Optionally, embodiments may also include an irrigation port on the proximal portion or the handle for applying irrigation fluid to the channel. In one embodiment, the suction port may be in fluid communication with a suction channel in an inner tube of the device, and wherein the irrigation port is in fluid communication with an irrigation channel comprising a space between an outer surface of the inner tube and an inner surface of the shaft of the device.

In some embodiments, the cutting member may include at least one rotating blade at least one stationary blade positioned relative to the rotating blade such that tissue is cut between the rotating blade and the stationary blade. In some embodiments, the cutting member may include multiple interdigitated cutters that rotate toward one another to shred tissue. In yet other alternative embodiments, the cutting member may include, but is not limited to, micro-shears, graspers and/or biopsy forceps. Some embodiments may include at least one tubular crown gear for driving the at least one cutting member. In some embodiments, the device may include two tubular crown gears coupled together with at least one intermediate gear disposed between them. For example, the intermediate gear may be disposed at a bend in the shaft located at an intersection of the proximal portion and the distal tip.

In some embodiments, the device may further include an energy transmission member coupled with the distal tip of the shaft for transmitting energy to the brain tumor. The energy transmitted by the energy transmission member may include, but is not limited to, radiofrequency, ultrasound, microwave, heat and laser energy.

In another aspect, a system for removing at least part of a brain tumor may include a mechanical tissue debrider. The debrider may include: a shaft having a proximal portion, a distal tip disposed at an angle relative to the proximal portion, and a channel extending from a distal end of the distal tip through at least part of the proximal portion; at least one moveable cutting member disposed at the distal end of the distal tip; a handle coupled with the proximal portion of the shaft; and an actuator coupled with the handle for actuating the at least one moveable cutting member. The system may further include suction tubing for connecting the handle to a source of suction.

Optionally, the system may also include an energy transmission member coupled with the distal tip of the shaft for transmitting an energy to the tissue. The energy may include, but is not limited to, radiofrequency, ultrasound, microwave, heat or laser energy. Some embodiments of the system may further include an irrigation port on the proximal portion of the shaft or the handle for applying irrigation fluid to the channel.

In another aspect, a method for removing at least part of a pituitary tumor in a patient may involve: advancing a distal end of a tissue cutter through a nostril and through the sphenoid sinus of the patient to contact a cutting member of the tissue cutter with the pituitary tumor; activating the cutting member to cut tissue from the pituitary tumor, wherein the cutting member does not extend laterally beyond the diameter of the tissue cutter shaft; and moving the cut pituitary tumor tissue through a channel of the shaft toward a proximal end of the tissue cutter. The tissue cutter may include a shaft having an outer diameter no greater than about 10 mm, which includes a distal shaft portion and a proximal shaft portion, and the distal shaft portion may be sharply angled relative to the proximal shaft portion.

In some embodiments, the method may involve, before contacting the pituitary tumor, forming an opening through the sphenoid sinus, and advancing the distal end of the tissue cutter through the opening. In some embodiments, the opening may be formed using the tissue cutter. In some embodiments, cutting the tissue may involve shredding the tissue. In some embodiments, moving the tissue may involve urging the tissue into the channel with cutting motion of the tissue cutter. In some embodiments, moving the cut tissue through the channel may further involve applying suction to the channel. In some embodiments, moving the cut tissue through the channel may further involve introducing fluid, via the tissue cutter, to an area at or near the distal end of the tissue cutter, where the applied suction moves at least some of the fluid proximally through the channel with the cut tissue.

In some embodiments, the cutting member may include at least one moveable blade and at least one stationary blade, and where cutting tissue comprises rotating the at least one rotating blade past the at least one stationary blade. In some embodiments, the cutting member comprises at least two interdigitated blades, and cutting tissue comprises rotating the two interdigitated blades toward one another. In other embodiments, the cutting member may include micro-shears, graspers and/or biopsy forceps. In some embodiments, the distal shaft portion may be angled relative to the proximal shaft portion by at least ______ degrees. In some embodiments, the proximal shaft portion may be curved. For example, the proximal shaft portion may include a gradual curve, a bayonet-shaped curve or both.

In some embodiments, the method may also include visualizing the cutting using a visualization device such as but not limited to a straight endoscope, an angled endoscope, a swing prism endoscope, a side viewing endoscope, a flexible endoscope, a CMOS digital camera, an ultrasound device or a scanning single fiber endoscope. In some embodiments, the visualization device may be incorporated into the tissue removal device. In some embodiments, the method may further include measuring an amount of the removed tissue by filtering the removed tissue from a stream of irrigation fluid. Some embodiments may further include measuring an amount of the removed tissue by determining motor torque in the tissue removal device during engagement of the device with the tissue and using the determined motor torque, a time period of tissue removal and/or a loading condition to approximate the amount of the removed tissue.

In some embodiments, the method may further involve monitoring a location of the tissue removal device during use, using a navigation system and at least one tracking feature on the device. In some embodiments, the method may involve collecting a sample of cut tissue, using a tissue capturing feature on the device, for use as a histological sample. Some embodiments of the method may further involve at least partially removing a blood clot from the patient through the shaft, where removing the blood clot includes breaking up the clot using the cutting member. In some embodiments, the tissue cutter may be coupled with an image guided or robotic surgical system during performance of at least part of the method. In some embodiments, the method may further involve protecting tissues not intended for treatment from contacting the cutting member during use of the device. In some embodiments, the method may further involve stimulating a portion of the pituitary tumor using a stimulation member at or near the distal end of the tissue removal device and deciding whether to cut the stimulated tissue, based on an observed response from the stimulation.

In another aspect, a device for removing at least part of a pituitary tumor may include: a shaft comprising a distal end, a proximal end, a distal shaft portion, a proximal shaft portion, a sharp bend at a juncture of the distal shaft portion and the proximal shaft portion, a channel extending from the distal end through at least part of the proximal portion, and an outer diameter no greater than about 10 mm; at least one moveable cutting member disposed at the distal end of the shaft such that, in use, the cutting member does not extend laterally beyond the outer diameter of the shaft; a handle coupled with the proximal portion of the shaft; an actuator coupled with the handle and the at least one cutting member to allow a user to activate the at least one cutting member via the handle; and at least one aperture on at least one of the handle or the proximal shaft portion and in fluid communication with the channel, for providing attachment to a source of suction force and/or withdrawal of cut tissue through the aperture. In some embodiments, the distal portion may have a length of no more than about 25 mm, and the bend may form an angle between the distal shaft portion and the proximal shaft portion of at least about 5 degrees. In some embodiments, the channel may extend from the distal end of the shaft to the at least one aperture.

In some embodiments, the device may include a suction port on the proximal portion or the handle for applying suction to the channel. In some embodiments, the device may include an irrigation port on the proximal portion or the handle for applying irrigation fluid to the channel. In another embodiment, the suction port may be in fluid communication with a suction channel in an inner tube of the device, and wherein the irrigation port is in fluid communication with an irrigation channel comprising a space between an outer surface of the inner tube and an inner surface of the shaft of the device.

In some embodiments, the moveable cutting member may include at least one rotating blade and at least one stationary blade positioned relative to the rotating blade such that tissue is cut between the rotating blade and the stationary blade. In an alternative embodiment, the moveable cutting member may include multiple interdigitated blades that rotate toward one another to shred tissue. In other alternative embodiments, the moveable cutting member may include, but is not limited to, micro-shears, graspers or biopsy forceps. In some embodiments, the device may include at least one tubular crown gear for driving the at least one cutting member. In some embodiments, the at least one tubular crown gear may include two tubular crown gears coupled together with at least one intermediate gear disposed between them. In some embodiments, the intermediate gear may be disposed at the bend in the shaft.

Some embodiments may further include an energy transmission member coupled with the distal tip of the shaft for transmitting energy to the pituitary tumor, and the energy transmitted by the energy transmission member may include, but is not limited to, radiofrequency, ultrasound, microwave, heat or laser energy. In some embodiments, the device may also include a visualization lumen coupled with an outer surface of the shaft, for holding at least a portion of an elongate visualization device. In some embodiments, the proximal portion of the shaft of the device may be curved. Some embodiments may further include at least one attachment member for attaching the device to an image guide or robotic surgical system. In some embodiments, the distal shaft portion may include a safety portion that extends along one side of the cutting member to prevent tissues not intended for treatment from contacting the cutting member during use of the device.

These and other aspects and embodiments of the invention will be described below in further detail, in relation to the attached drawings.

### BRIEF DESCRIPTION OF THE DRAWINGS

FIGS. 1-4 are various cross-sectional views of a human head, illustrating the location of the pituitary gland and pituitary tumors;

FIG. 5 is a perspective view of a portion of a human head, illustrating a typical surgical access pathway to a pituitary tumor;

FIG. 6 is a perspective view of a human head, partially cut away to illustrate a typical surgical access pathway to a pituitary tumor;

FIGS. 7A-7D are various views of portions of a human head, illustrating the position and surgical access route to one example of a pituitary tumor, using a tissue cutter/micro-debrider device, according to one embodiment;

FIGS. 8A-8C are various views of portions of a human head, illustrating the position and surgical access route to one example of a pituitary tumor, using a tissue cutter/micro-debrider device, according to an alternative embodiment;

FIGS. 9A and 9B are two views of portions of a human head, illustrating the position and surgical access route to one example of a pituitary tumor, using a tissue cutter/micro-debrider device, according to another alternative embodiment;

FIG. 10 is a side view of a human head, illustrating the position and surgical access route to one example of a pituitary tumor, using a tissue cutter/micro-debrider device, according to another alternative embodiment;

FIGS. 11A and 11B are perspective and side views, respectively, of a tissue cutter/micro-debrider device for removing brain tumor tissue, according to one embodiment;

FIGS. 12A and 12B are perspective and side views of the tissue cutter/micro-debrider device of FIGS. 11A and 11B;

FIGS. 13A and 13B are perspective and side views, respectively, of a tissue cutter/micro-debrider device for removing brain tumor tissue, according to an alternative embodiment with a differently angled shaft;

FIGS. 14A and 14B are perspective and side views, respectively, of a tissue cutter/micro-debrider device for removing brain tumor tissue, according to another alternative embodiment with a differently angled shaft;

FIGS. 15A and 15B are perspective and side views, respectively, of a tissue cutter/micro-debrider device for removing brain tumor tissue, according to another alternative embodiment with a differently angled shaft;

FIG. 16 is a side view of a tissue cutter/micro-debrider device with interdigitated cutters for performing a tongue reduction procedure for sleep apnea, according to one embodiment;

FIG. 17 is a perspective view of a tissue cutter/micro-debrider device with a concentric cutter for performing a tongue reduction procedure for sleep apnea, according to an alternative embodiment;

FIG. 18 is a perspective view of an angled cutter head of the micro-debrider device of FIG. 16;

FIG. 19 is a perspective view of an angled cutter head of the micro-debrider device of FIG. 17;

FIG. 20 is a close-up, perspective view of a portion of a tissue cutter/micro-debrider device, including the sharp distal bend, according to one embodiment;

FIG. 21 is a close-up view of the portion of the tissue cutter/micro-debrider device of FIG. 20, from a different perspective;

FIGS. 22 and 22A-22F are side views of various embodiments of a tissue cutter/micro-debrider device, each having a different shaft configuration, according to various alternative embodiments;

FIG. 23 is a side view of a portion of a tissue cutter/micro-debrider device, illustrating various components for forming a gradual proximal bend in the shaft of the device, according to various alternative embodiments;

FIG. 24 includes various views of various embodiments of a laser cut pattern for forming a gradually curving proximal portion of a shaft of a tissue cutter/micro-debrider device, according to various alternative embodiments;

FIGS. 25A-25C are side views of a gradually curving proximal portion of a shaft of a tissue cutter/micro-debrider device, illustrating one method for construction the gradual curve, according to one embodiment;

FIG. 26 is a side view of a portion of a shaft of a tissue cutter/micro-debrider device, illustrating a hinge and movement about the hinge, according to one embodiment;

FIG. 27 is a perspective view of three embodiments of a distal end of a tissue cutter/micro-debrider device, each including electrodes, according to various embodiments;

FIG. 28 is a perspective view of three embodiments of a distal end of a tissue cutter/micro-debrider device, each including at least one electrode, according to various embodiments;

FIG. 29 is a perspective view of two embodiments of a distal end of a tissue cutter/micro-debrider device, each including electrodes, according to various embodiments;

FIG. 30 is a perspective view of two embodiments of a distal end of a tissue cutter/micro-debrider device, each including two extending, curved electrodes housed in sheaths, according to various embodiments;

FIG. 31 is a perspective view of two embodiments of a distal end of a tissue cutter/micro-debrider device, each including extending, looped electrodes housed in sheaths, according to various embodiments;

FIG. 32 is an exploded view of the distal end of a tissue cutter/micro-debrider device, according to one embodiment;

FIG. 33 is a perspective, partially exploded view of the distal end of a tissue cutter/micro-debrider device, according to an alternative embodiment;

FIG. 34 is a perspective view of a cutting member of a tissue cutter/micro-debrider device, according to one embodiment;

FIG. 35 is a perspective view of a cutting member of a tissue cutter/micro-debrider device, according to one embodiment;

FIG. 36 is a perspective view of a cutting member of a tissue cutter/micro-debrider device, according to an alternative embodiment;

FIG. 37 is a perspective view of a distal end of a tissue cutter/micro-debrider device, according to one embodiment;

FIG. 38 is a perspective view of distal ends of two tissue cutter/micro-debrider devices, according to alternative embodiments;

FIG. 39A is a perspective view of a distal end of a tissue cutter/micro-debrider device, according to another alternative embodiment;

FIG. 39B is a side cross-sectional view of the device end shown in FIG. 39A;

FIG. 40 is a top view and magnified top view of the device end shown in FIG. 39A;

FIGS. 41A and 41B are perspective views of an articulating opposable end effector configured as micro-shears for a micro-mechanical tissue removal tool, according to one embodiment;

FIGS. 42A and 42B are perspective views of an articulating opposable end effector configured as graspers for a micro-mechanical tissue removal tool, according to one embodiment;

FIGS. 43A and 43B are perspective views of an articulating opposable end effector configured as biopsy forceps for a micro-mechanical tissue removal tool, according to one embodiment;

FIGS. 44A-44C are perspective views of an articulating opposable end effector with a wrist for a micro-mechanical tissue removal tool, according to one embodiment;

FIGS. 45A and 45B are perspective and top views, respectively, of a an articulating opposable end effector with a wrist and an additional articulation point for a micro-mechanical tissue removal tool, according to one embodiment;

FIG. 46 is a perspective view of a distal end of a tissue cutter/micro-debrider device, according to another alternative embodiment;

FIGS. 47A and 47B are perspective and side views, respectively, of a tissue cutter/micro-debrider device, according to another alternative embodiment;

FIG. 48 is a side view of a portion of a human head and a tissue cutter/micro-debrider device including a speculum, according to another alternative embodiment; and

FIG. 49 is a side view of a portion of a human head and a tissue cutter/micro-debrider device including image guidance members, according to another alternative embodiment.

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

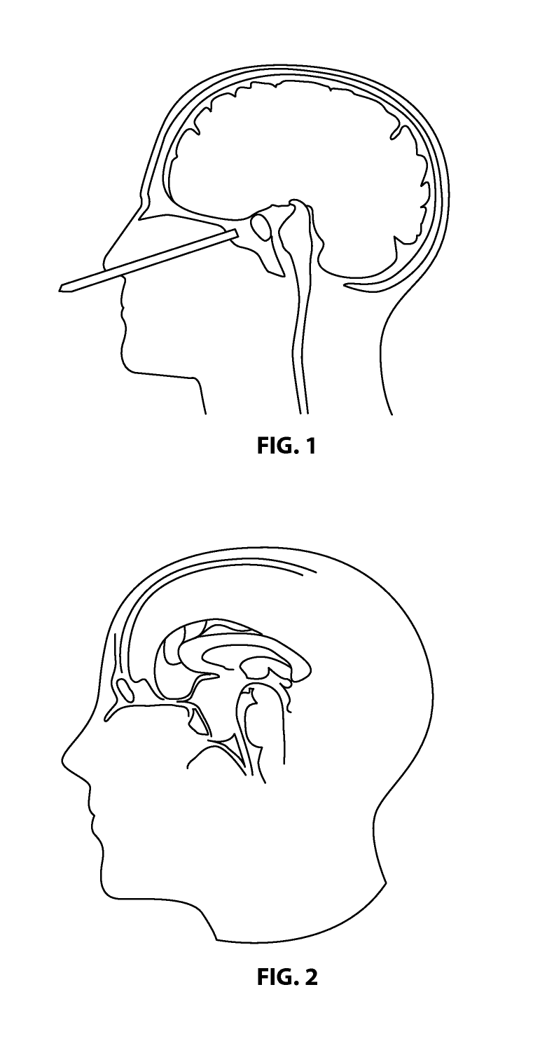

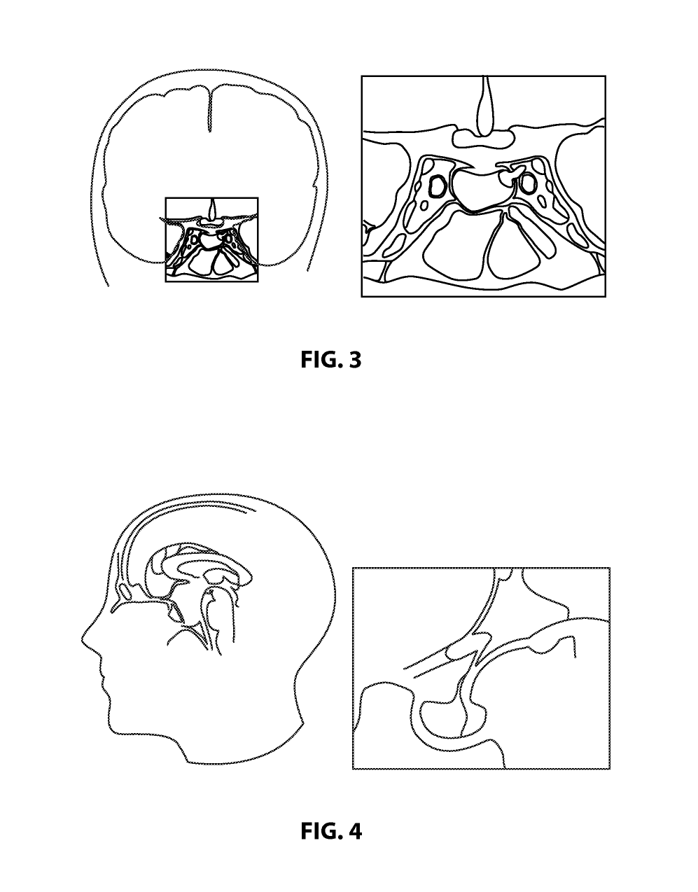

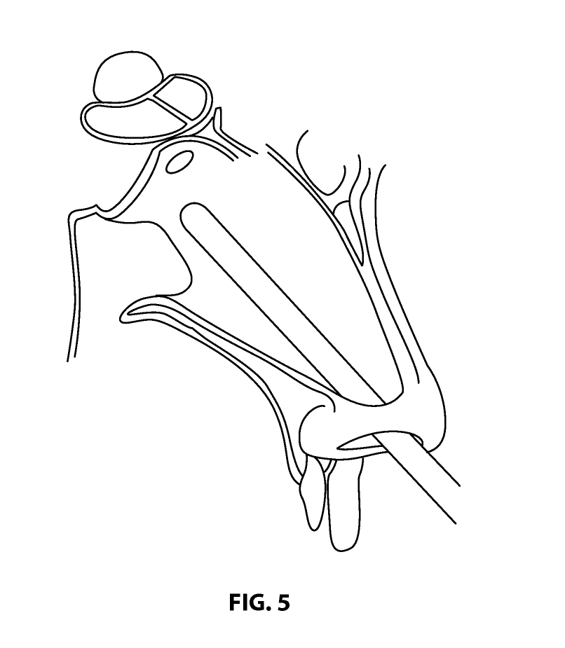

FIGS. 1-6 are various anatomical drawings of portions of human heads, illustrating the location of pituitary tumors. FIG. 1 is a sagittal cross section of a human head, illustrating the typical, surgical access route to a pituitary tumor, through one or both nostrils and the sphenoid sinus. FIG. 2 is also as sagittal cross section, illustrating some of the anatomical structures surrounding a pituitary tumor. FIG. 3 includes a coronal cross section of a human head and a close-up view of a portion of the coronal cross section. These views show the proximity of the pituitary gland to the internal carotid arteries and the optic nerve—two important structures that may be accidentally damaged during a pituitary tumor removal procedure. FIG. 4 includes a sagittal cross second of a human head and a close-up view of a portion of the sagittal cross section. These views show the proximity of the pituitary gland to the optic nerve and the hypothalamus. FIG. 5 is a perspective view/partial cross section of a face, showing an endoscope advanced through a nostril to illustrate the typical surgical access route to a pituitary tumor through a nostril and the sphenoid sinus.

FIG. 6 is a side/partial cross section of a face, showing an endoscope and a side-biting rongeur removal device advanced through a nostril, thus also illustrating the typical, surgical access route to a pituitary tumor. Conventional surgical tools for pituitary tumor removal are straight and manually operated. Because the tools are straight, they can only be used to remove target tissue that is directly in front of the leading edge. If the target tissue is off to the left or right of the tool axis, safely removing the tissue without damaging non-target tissue can be very difficult or impossible. Non-target, critical structures include the optic nerve, the carotid artery and the pituitary gland itself. Additionally, when surgical tools have been developed in the past for applications such as pituitary tumor removal, they typically were not able to be made with a sharp bend near the distal end. This is due to the internal mechanisms used to drive the cutting mechanism at the distal end and the inability to curve those mechanisms around a sharp bend. Attempts to make a curved surgical tissue removal tool often resulted in a tool with a gradual curve and/or a relatively wide diameter, which would not work well when a nostril access path is used. Adding to the difficulty of pituitary tumor removal is the fact that the tumor is removed through a small opening deep within the nasal cavity, through the sphenoid sinus. It is often difficult to view the surgical site, and typically an endoscope must be used along with the tissue removal tool for visualization. Thus, the tool must have a relatively small outer diameter to work in the small surgical space along with an endoscope.

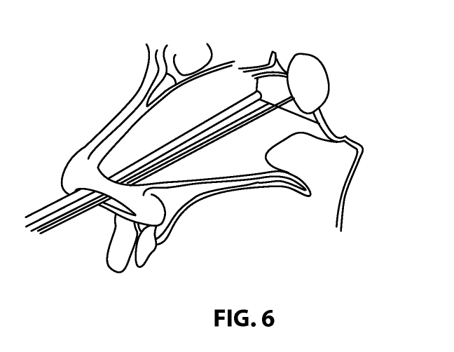

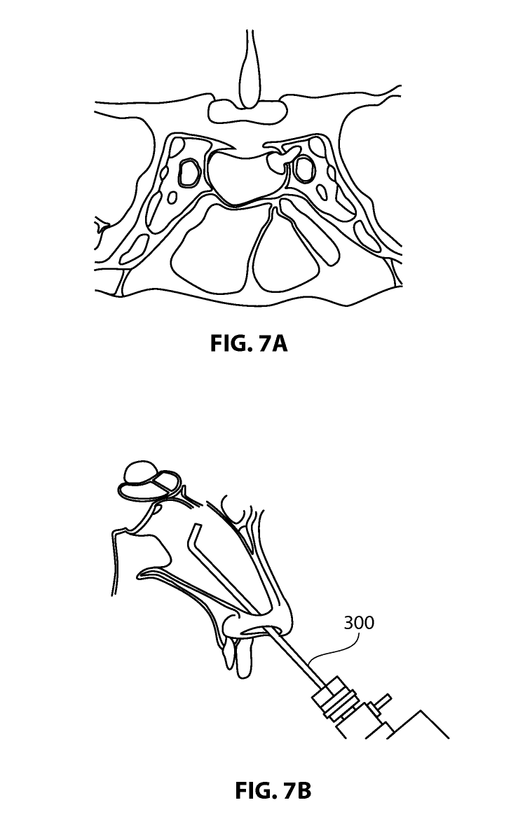

FIGS. 7A-7D illustrate an alternative method for accessing a pituitary tumor. FIG. 7A is a cross sectional view of a portion of a human head, showing a pituitary tumor encroaching on the cavernous sinus and contacting the posterior aspect of one of the internal carotid arteries. Obviously, this example of a pituitary tumor would be difficult or impossible to remove with a straight, side-cutting surgical device advanced through the nostril, without seriously jeopardizing the internal carotid artery.

As illustrated in FIGS. 7B-7D, in one embodiment, an improved surgical tissue cutter 300 may be advanced into a nostril and through the sphenoid sinus to contact a pituitary tumor. In this embodiment, as will be described in greater detail below, the shaft of tissue cutter 300 includes a sharp bend (or “joint” or “angle”) near its distal end, thus forming a sharp angle between a distal portion and a proximal portion of the shaft. In some embodiments, this bend or angle is fixed, while in other embodiments the bend or angle may be adjustable. The terms “sharp bend” and “sharp angle” are used herein to mean that the bend or angle occurs at a distinct point along the length of the shaft, in contrast to a gradual bend or curve that might be found in another instrument or, as described below, in the proximal portion of the shaft. In various embodiments, the sharp bend may form an angle between the distal portion and the proximal portion of between about 1 degree and about 90 degrees, and ideally the angle will be between about 45 degrees and about 90 degrees. In prior art tissue cutting devices, such a sharp bend within a small-diameter device was not feasible, because there was no suitable way to drive the cutting mechanism at the distal end of the device.

Tissue cutter 300 contacts the pituitary tumor with front-facing cutting member(s), rather than side-facing cutting members of prior art devices. At the same time, it still faces partially sideways, due to the sharp bend in the shaft of the device. This configuration allows a user to remove tumor tissue around a tight corner, sometimes in areas that are difficult to see, as in the example of the tumor shown in FIGS. 7A-7D. As will be described below in greater detail, tissue cutter 300 is generally a micro-mechanical tissue debrider that cuts very small pieces of tissue and moves the tissue proximally through the inner channel of the shaft of cutter 300. Cut tissue can be moved proximally simply by the action of the cutting member(s) or with suction force applied to cutter 300 or both. In some cases, cutter 300 may include an irrigation function as well, so that irrigation fluid may be introduced to the tissue removal site, and suction applied by tissue cutter 300 may then be used to remove the introduced fluid and cut tissue. When the pituitary tumor has thus been removed, tissue cutter 300 may be withdrawn out the nostril.

In some embodiments, the amount of the removed tissue is approximated by determining motor torque in the tissue removal device during engagement of the device with the tissue. The motor torque, a time period of the tissue removal and/or a loading condition may be used to approximate the amount of the removed tissue.

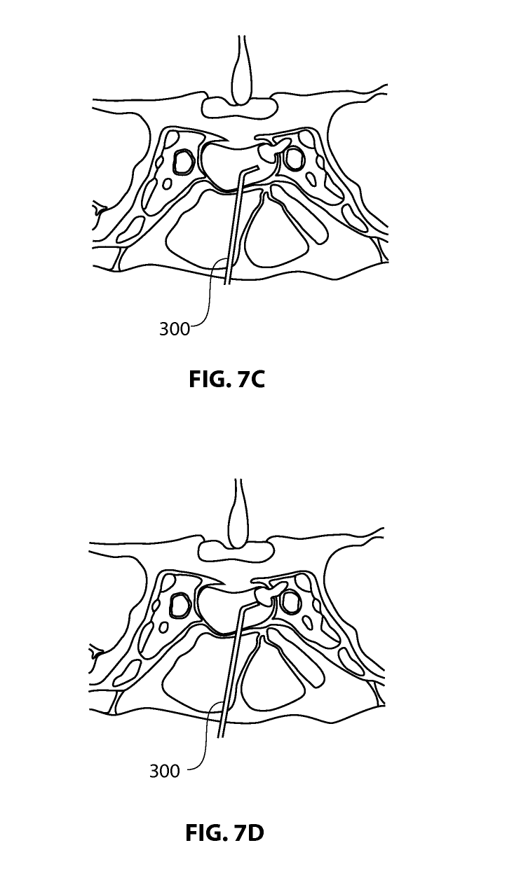

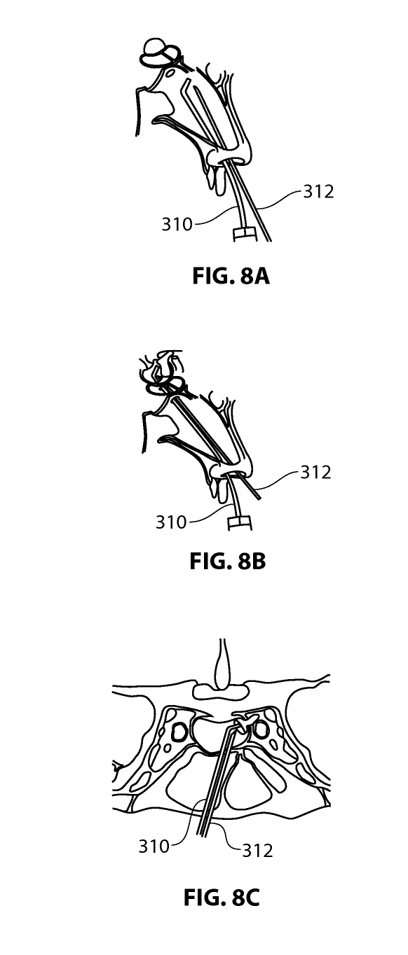

Referring now to FIGS. 8A-8C, another embodiment of a method for accessing a pituitary tumor is illustrated. In this embodiment, a tissue cutter 310 includes a gradually curved proximal portion. This gradual curve may facilitate advancement of an endoscope 312 into the nose via the same or opposite nostril to the surgical site, as illustrated in FIGS. 8A-8C. As seen best in FIG. 8B, tissue cutter 310 and endoscope 312 may be advanced into place for removing tissue without conflicting with one another and while the handle of tissue cutter 310 remains out of the way of endoscope 312. In this embodiment, endoscope 312 tracks along the “inside” (i.e., the side closest to the tumor) of tissue cutter 310.

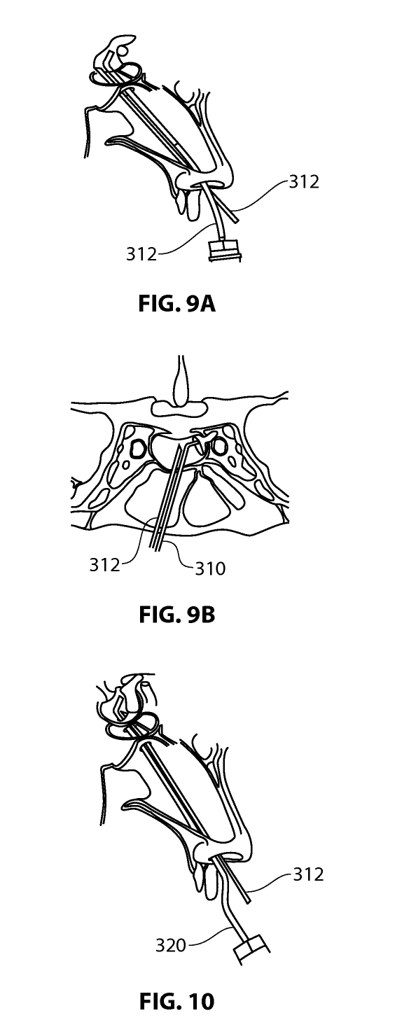

FIGS. 9A and 9B illustrate a slightly different method for accessing a pituitary tumor using tissue cutter 310 and endoscope 312. In this embodiment, tissue cutter 310 and endoscope 312 cross over one another (from a side view) so that the distal end of endoscope 312 is located “outside” (or behind, i.e., the side away from the tumor) the distal end of tissue cutter 310. This view from behind may be advantageous in some scenarios. In at least one embodiment, as shown in FIGS. 8A-8C, 9A and 9B, the same tissue cutter 310 and endoscope 312 may be used to achieve either access configuration discussed here.

With reference now to FIG. 10, in another embodiment, a tissue cutter 320 may include a bayonetted proximal shaft portion, rather than the gradually curved proximal shaft portion of the earlier embodiments. In the same way as the gentle curve, the bayonetted section may facilitate advancement of tissue cutter 320 and endoscope 312 through the nose and to a surgical location for pituitary tumor removal.

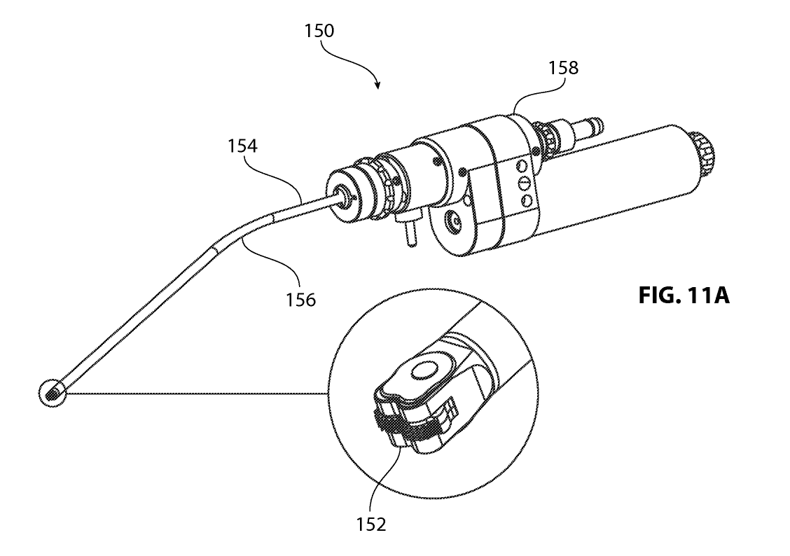

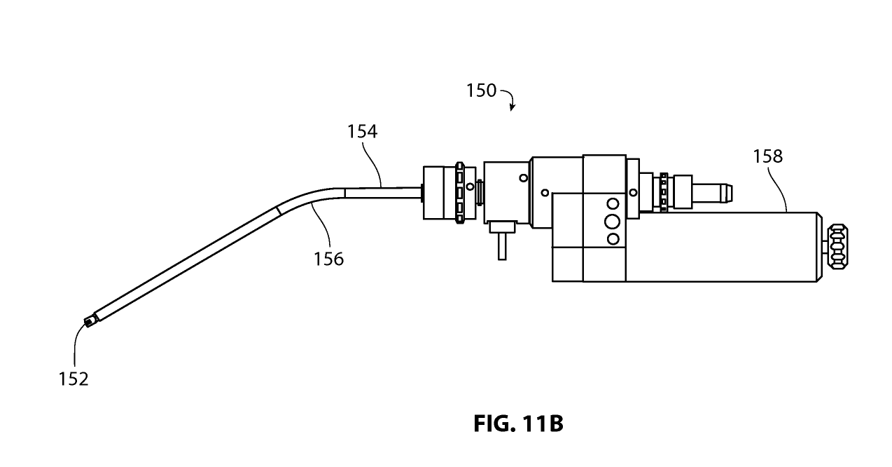

FIGS. 11A and 11B illustrate one embodiment of a micro-debrider device 150 (or “tissue cutter”) for removal of pituitary tumor tissue (or other brain tumor tissue in alternative embodiments. In the present disclosure, “tissue cutter” will usually, but not necessarily, be used to refer to an entire tissue cutting device (or “micro-debrider device”). Generally, tissue cutter 150 includes a handle 158, a shaft 154 and a tissue cutting member 152 (or “micro-debrider”) at the distal end of shaft 154. In the present disclosure, “cutting member” or “micro-debrider” will usually, but not necessarily, be used to refer to the cutting elements at the distal end of a tissue cutter/micro-debrider device. Shaft 154 includes a bend 154 somewhere along its length, which may form an angle between a proximal portion and a distal portion of shaft 154 from greater than 0 degrees to about 90 degrees. As discussed above and described further below, in many embodiments, bend 154 may be sharper and may be positioned closer to the distal end of device 150 than is shown in the embodiment of FIGS. 11A and 11B. In some embodiments, shaft 154 may include one or more proximal curves or bends 156 and also a distal bend (not illustrated in FIGS. 11A and 11B). Tissue cutter 152, in this embodiment, includes multiple interdigitating blades that rotate toward one another to shred tissue. In general, tissue cutter 152 is so small that fiber-by-fiber tissue removal can be accomplished, thus allowing a surgeon to remove brain tumor tissue accurately and safely while helping prevent damage to nearby tissues. In various embodiments, any of a large number of different types of tissue cutters may be incorporated onto the distal end of micro-debrider device 150. In some embodiments, the distal shaft portion includes a safety portion that extends along one side of the cutting member to prevent tissues not intended for treatment from contacting the cutting member during use of the device.

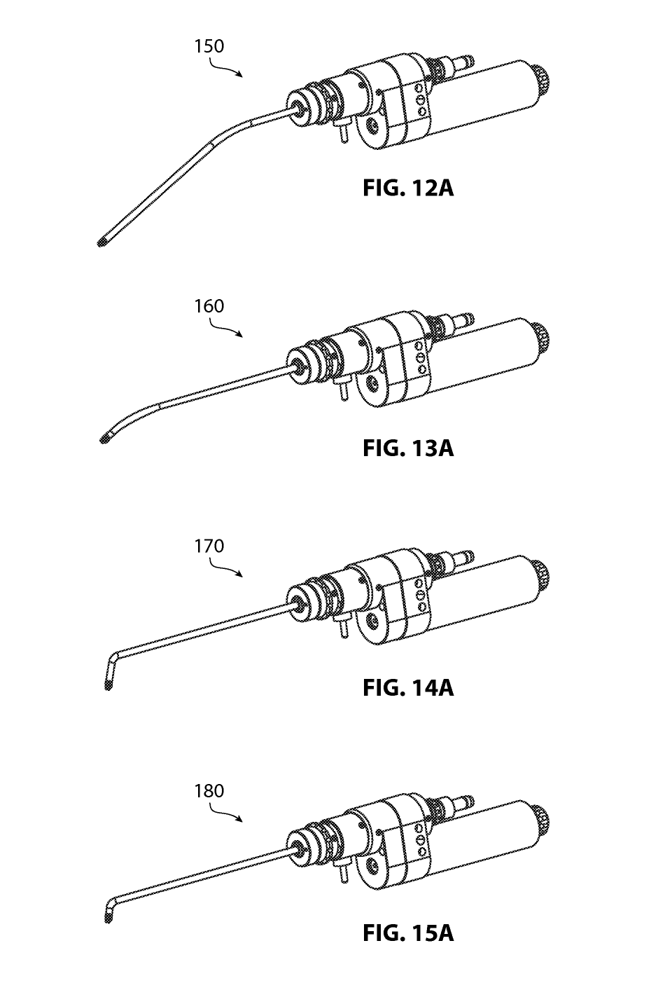

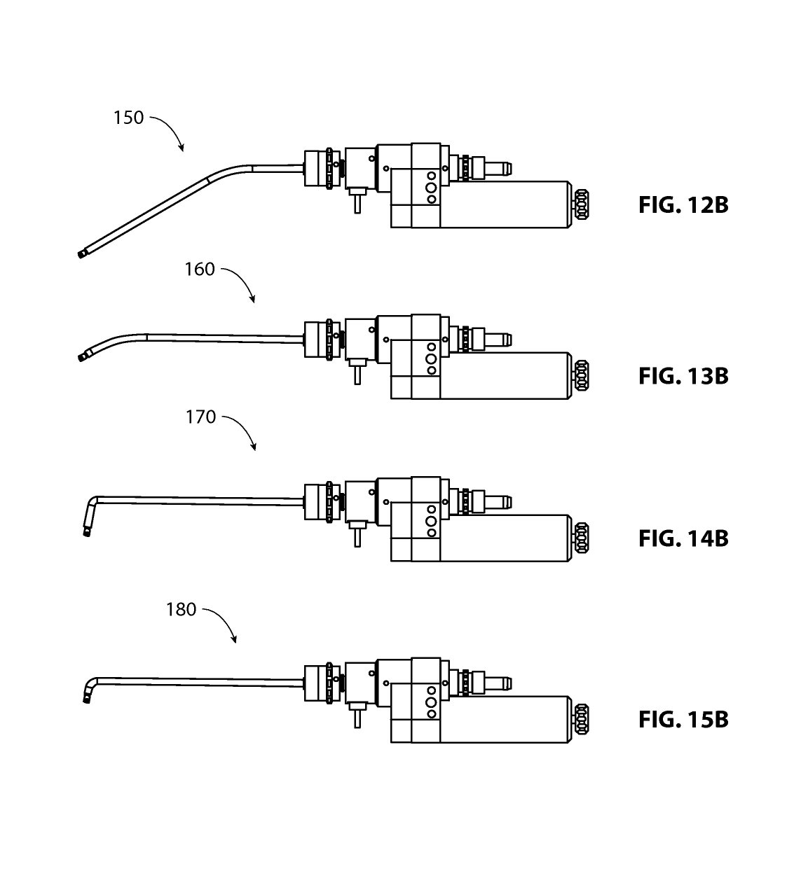

FIGS. 12A and 12B are additional views of tissue cutter 150. FIGS. 13A-15B illustrate other, alternative embodiments of a tissue cutter/micro-debrider device 160, 170, 180, each having a shaft with a differently angled and/or differently located bend. In other alternative embodiments, any other suitable location and angle of a bend in a shaft may be used. In many embodiments, the bend angle and location will be configured to provide a desired tool for approaching a tumor located in a particular part of the brain. The angle and location of the bend will typically allow for visualization of the tumor and/or at least some of the surrounding anatomy over the top of the tissue removal device or from below the tissue device. For example, the tissue cutters 170 and 180 (FIGS. 14A-15B), each including a sharp bend in the distal portion of the shaft, may be particularly advantageous for accessing and removing pituitary tumors through a nostril and a sphenoid sinus. Other embodiments may be used for open brain surgery approaches, acoustic neuroma removal, and/or other brain surgeries. In fact, although the present disclosure focuses primarily on devices and methods for accessing and removing pituitary tumors, in a number of embodiments, devices and methods described herein may be used to access and remove other brain tumors.

Any of the embodiments described above or below may include any suitable end effector. Generally, these end effectors may be referred to as “cutting members” or “micro-debriders,” though in some embodiments, the end effectors may not cut tissue (for example, graspers or forceps). In the embodiments show above, the end effector is a tissue shredder having interdigitating blades that rotate toward one another to cut tissue. In an alternative embodiment, the end effector may be a concentric cutter, including at least one rotating blade and one stationary blade. In other embodiments, micro-shears (or “scissors”), graspers, biopsy forceps or other tools may be the end effectors. In this disclosure, the terms “cutting member” and “micro-debrider” are used generally and interchangeably to refer to any end effectors of the small-diameter devices described herein that cut tissue.

Additionally, various alternative embodiments may include any suitable combination of handle, shaft length, shaft bend angle and the like. These combinations may be used with any suitable micro-debrider or other end effector in various embodiments.

Optionally, any embodiment of the brain tissue removal tools described herein may include features (or an entire system) for providing navigation. For example, the device may include one or more fiducials, coils or other tracking devices, and may use and/or be compatible with any suitable infrared, radiofrequency, CT, MRI or other system. With such tracking/navigation systems, the cutter/end effector, shaft and/or handle may be tracked.

In some embodiments, the brain tumor removal device may also include features for mapping the brain tumor. For example, such embodiments may include an RF or other stimulator for stimulating portions of brain or tumor to determine when it is safe to cut tissue. Additionally, some embodiments may include a feature for collecting cut tissue samples for histology, for example an aperture or other collection member in the shaft and/or handle of the device.

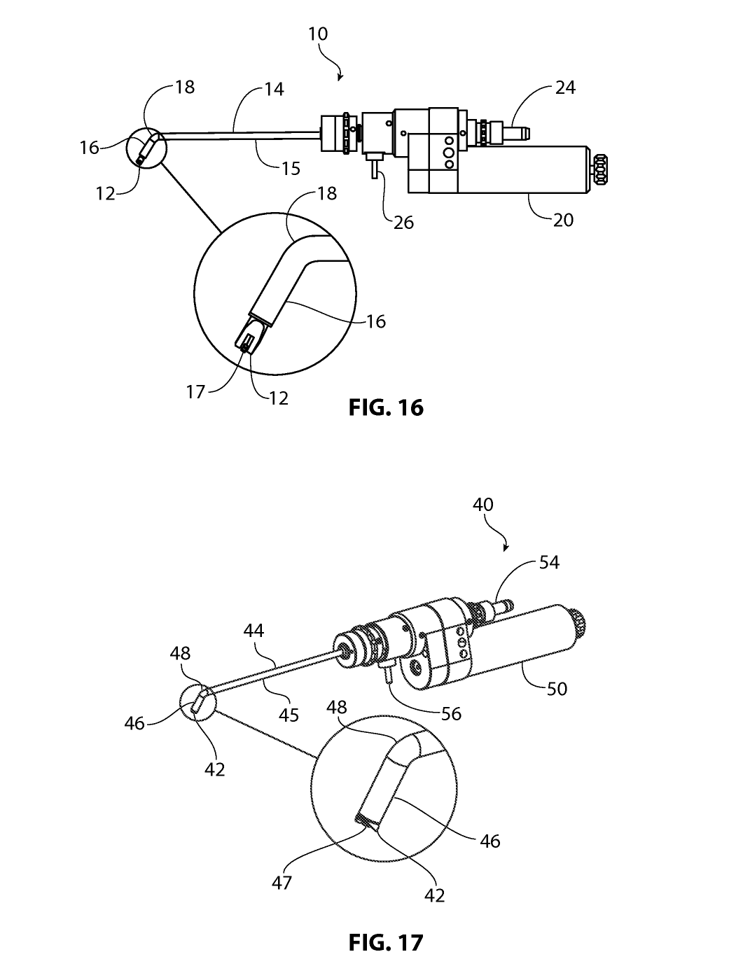

Referring now to FIG. 16, one embodiment of a tissue cutter/micro-debrider device 10 is shown in more detail. Many of the features and aspects of micro-debrider device 10 are described in greater detail in U.S. patent application Ser. No. 13/007,578 (Pub. No. 2012/0109172), entitled “Selective Tissue Removal Tool for Use in Medical Applications and Methods for Making and Using,” filed on Jan. 14, 2011, which is hereby incorporated by reference in its entirety. Micro-debrider device 10 may include a handle 20 and a shaft 14. Shaft 14 may include a proximal shaft portion 15, a distal shaft portion 16 (or “distal tip”), and a bend 18 at the intersection of proximal portion 15 and distal portion 16. As shown in greater detail in the close-up view, distal tip 16 includes a distal end 12 and a cutting member 17 at distal end 12. In this embodiment, cutting member 17 includes multiple, interdigitated blades, which will be described further below.

Handle 20 may include, in some embodiments, a suction port 24 and/or an irrigation port 26 for coupling handle 20 with a source of suction and/or irrigation, respectively. Ports 24, 26 are in fluid communication with one or two channels extending through shaft 14. In some embodiments, for example, shaft 14 may include a suction channel and an irrigation channel. In alternative embodiments, shaft 14 may include one common suction/irrigation channel. In one embodiment with two channels, device 10 may include an inner shaft (not visible in FIG. 12) and an outer shaft 14, with the middle bore of the inner shaft being used as a suction lumen and a space between the outer surface of the inner shaft and the inner surface of outer shaft 14 being used as an irrigation lumen. In an alternative embodiment, the opposite configuration for suction/irrigation may be used.

In general, the outer diameter of shaft 14 may be relatively quite small, since cutting member 17 and the mechanical elements used to drive it are also quite small. This small outer shaft diameter may facilitate use of device 10 within the mouth, nose or other body cavity. The angle of bend 18 and the length of distal portion 16 may also be designed to facilitate usability. In some embodiments, for example, shaft 14 may have an outer diameter of between about 1 mm and about 10 mm, distal portion 16 may have a length of between about 1 mm and about 25 mm, and bend 18 may form an angle of between about 1 degree and about 90 degrees. Even more ideally, in some embodiments, the outer diameter of shaft 14 may be between about 2 mm and about 4 mm, and bend 18 may form an angle of between about 30 degrees and about 90 degrees.

In various alternative embodiments, bend 18 may be fixed or adjustable. In the embodiments shown and described in FIG. 12 and other figures herein, bend 18 is generally fixed. However, in alternative embodiments, bend 18 may be manually adjustable to adjust the angle or may be mechanically adjustable by the device itself.

Referring now to FIG. 17, an alternative embodiment of a tissue cutter/micro-debrider device 40 is shown. As with previously described embodiments, micro-debrider device 40 may include a handle 50 and a shaft 44. Shaft 44 may include a proximal portion 45, distal tip 46 and bend 48 at the intersection of proximal portion 45 and distal tip 46. As shown in greater detail in the close-up view, distal tip 46 includes distal end 42 and a cutting member 47 at distal end 42. In this embodiment, cutting member 47 includes a rotating blade that rotates past a stationary blade, as will be described further below. Handle 50 may include, in some embodiments, a suction port 54 and/or an irrigation port 56, for coupling handle 50 with a source of suction and/or irrigation, respectively. Ports 54, 56 are in fluid communication with one or two channels extending through shaft 44.

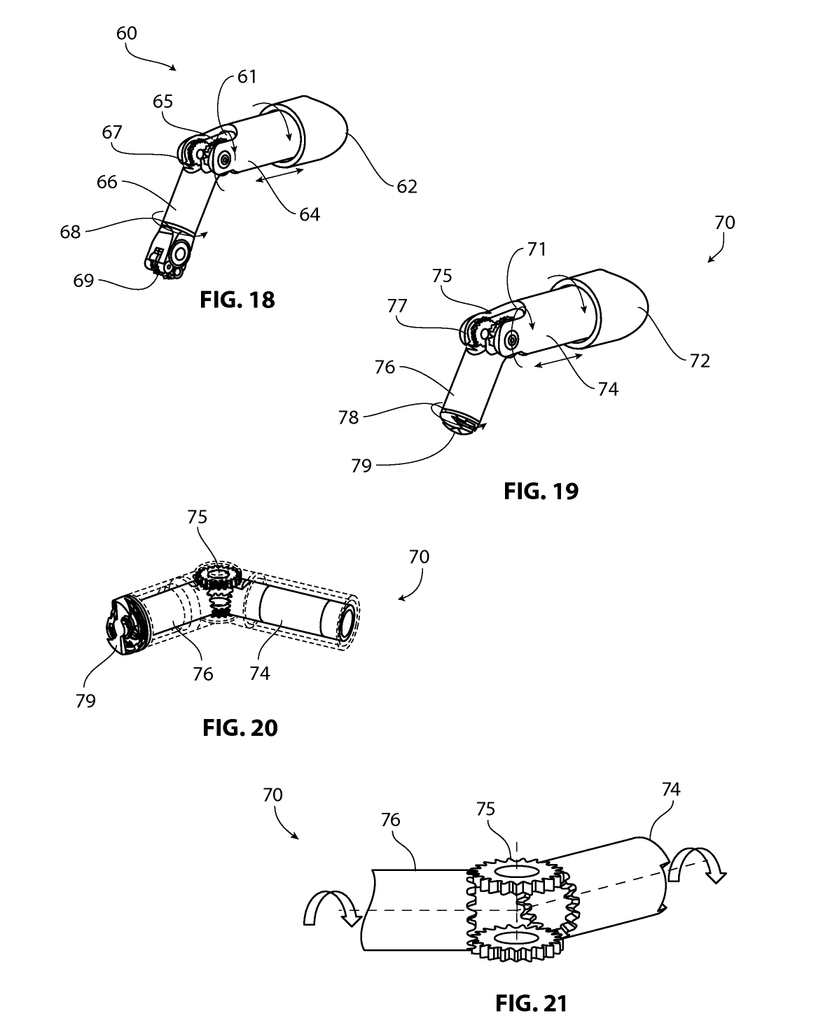

Referring now to FIG. 18, a portion of one embodiment of a tissue cutter/micro-debrider device 60 is illustrated, showing the junction between the proximal portion and the distal portion of tissue cutter 60. In this embodiment, a first arm 66 (also referred to as the “distal portion” or “distal tip” herein) is connected to a second arm 64 (or “inner shaft”) via a middle gear 65. A proximal support 62 (or “outer shaft”) surrounds at least part of second arm 64. First arm 66 is coupled with a tissue shredder 69 via a wrist 68, which allows shredder 69 to rotate relative to first arm 66. In this embodiment, movement and/or adjustments may occur at wrist 68, and the angle between first arm 66 and second arm 64 is fixed. In various alternative embodiments, first arm 66 and second arm 64 may be adjustable, relative to one another. Such adjustments may be carried out via mechanisms within device 60 or alternatively by manually adjusting device 60 with the hands or an adjustment tool. In this embodiment, a first crown gear 67 resides on the proximal end of a first inner drive tube inside first arm 66, and a second crown gear 61 resides on the distal end of a second inner drive tube inside second arm 64. Second crown gear 61 rotates to turn middle gear 65, and middle gear 65 rotates first crown gear 67, which turns the blades of tissue shredder 69 via the first inner drive tube. In the embodiment shown, two sets of blades rotate in opposite directions toward one another to cut (or “shred” or “tear”) tissue and also to urge the cut tissue into device 60. The configuration of first crown gear 67, middle gear 65 and second crown gear 61 allows device 60 to have a very sharp bend and a very small diameter, while still providing for effective driving of the blades of tissue shredder 69. Prior art blade driving mechanisms typically do not allow for such sharp bends and/or small diameters, because conventional drive mechanisms are not able to drive distal actuated cutters through a tight bend.

Referring now to FIG. 19, a portion of another embodiment of a tissue cutter/micro-debrider device 70 is illustrated, again showing the juncture of the proximal and distal portions of tissue cutter 70. In this embodiment, a first arm 76 (or “distal portion” or “distal tip”) is connected to a second arm 74 (or “inner shaft”) via a middle gear 75. A proximal support 72 (or “outer shaft”) surrounds at least part of second arm 74. First arm 76 is coupled with a concentric cutter 79 via a wrist 78, which allows concentric cutter 79 to rotate relative to first arm 76, while in some embodiments first arm 76 and second arm 74 are fixed relative to one another. As with the previously described embodiment, a first crown gear 77 resides inside first arm 76, and a second crown gear 71 resides inside second arm 74. Second crown gear 71 rotates to turn middle gear 75, and middle gear 75 rotates first crown gear 77, which turns the blade of concentric cutter 79.

FIGS. 20 and 21 are additional drawings of the portion of tissue cutter 70 from FIG. 19. FIG. 21 illustrates, with arrows, how motion is transmitted along the shaft via the crown gears.

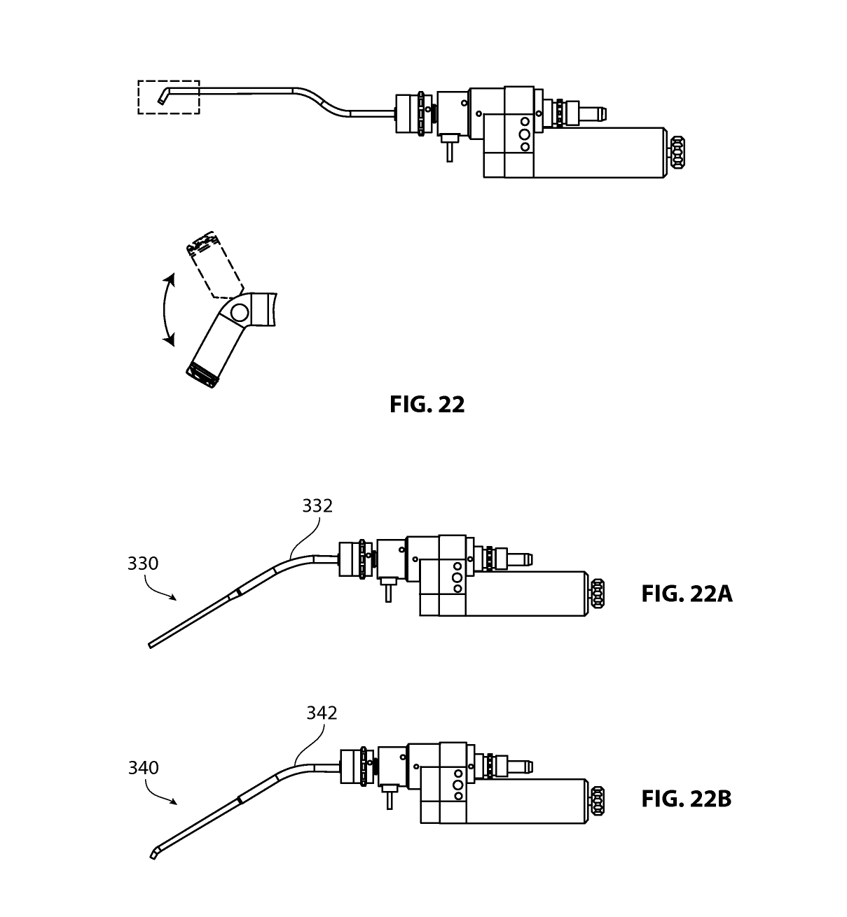

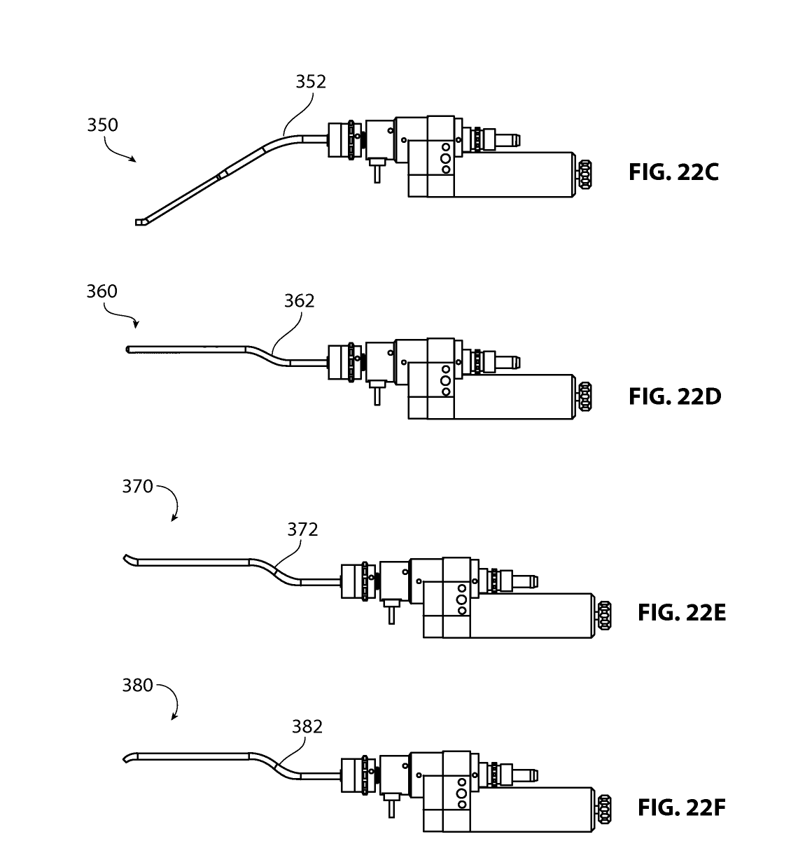

FIGS. 22A-22F illustrate various alternative embodiments of a tissue cutter/micro-debrider device, each having a different shaft configuration. The embodiments illustrated in FIGS. 22A-22C are tissue cutters 330, 340, 350 with a proximal shaft portion that includes a gradual curve 332, 342, 352, respectively. FIGS. 22D-22F are tissue cutters 360, 370, 380 with a proximal shaft that includes a bayonetted curve 362, 372, 382, respectively. As illustrated in these various figures, any of a number of curve configurations in the proximal shaft portion may be combined with any of a number of distal curve configurations.

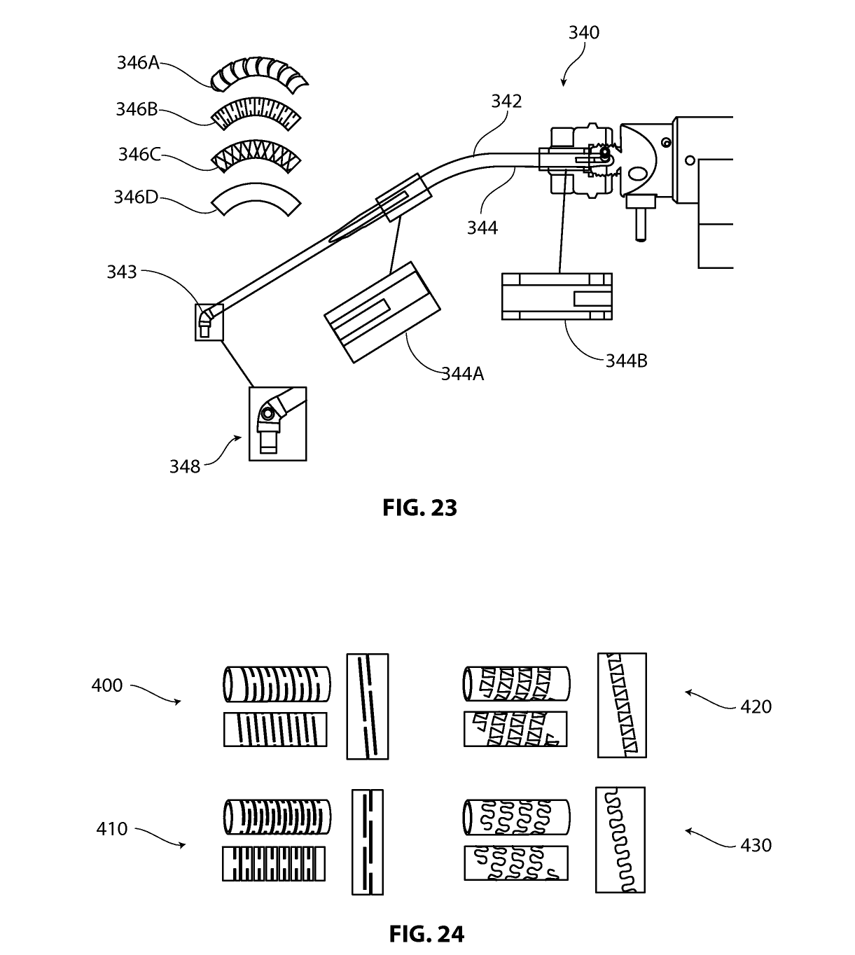

FIG. 23 illustrates the embodiment of tissue cutter/micro-debrider device 340, from FIG. 22B, in greater detail. As discussed, tissue cutter 340 includes a shaft 344, which may include a proximal, gradual curve 342 and a distal, sharp bend 343. As used herein, “proximal shaft portion” means the portion of shaft 344 proximal to distal bend 343, and “distal shaft portion” means the portion 348 of shaft 344 distal to distal bend 343. Proximal curve 342 may be configured in the same direction or the opposite direction as distal bend 343, in various embodiments, and may take up all or a portion of the length of the proximal shaft portion. In some embodiments, the proximal curve and the distal curve lie in a common plane, as shown in FIG. 23. In other embodiments (not shown), the proximal curve and the distal curve lie in different planes which may be orthogonal to one another. In some embodiments, proximal curve 342 may be formed using various flexible, continuous sections, as illustrated in the magnified boxes labeled 344a and 344b. These magnified views show the beginning and end points for the flexible inner shaft. In various embodiments, the continuous sections may include links 346a, laser cut sections 346b, woven mesh 346c, continuous polymer 346d, or some combination thereof. Again, proximal curve 342 may have any suitable angle, shape and length, based on whatever the desired properties of the overall tissue cutter 340 and the tumor to be addressed. In some embodiments designed for resecting portions of the pituitary gland, the distal end is angled between 70 and 170 degrees from the axis of the adjacent (proximal) shaft.

Referring to FIG. 24, various patterns 400, 410, 420, 430 for laser cutting a shaft of a tissue cutter to form the proximal curve are illustrated. As evident from these patterns, any of a number of suitable laser cut patterns 440, 410, 420, 430 (including patterns not illustrated) may be used to form the curve. In some embodiments, the radius of curvature of the proximal bend is between 1 and 3 inches. In some embodiments, the proximal angle is between 10 and 90 degrees from straight.

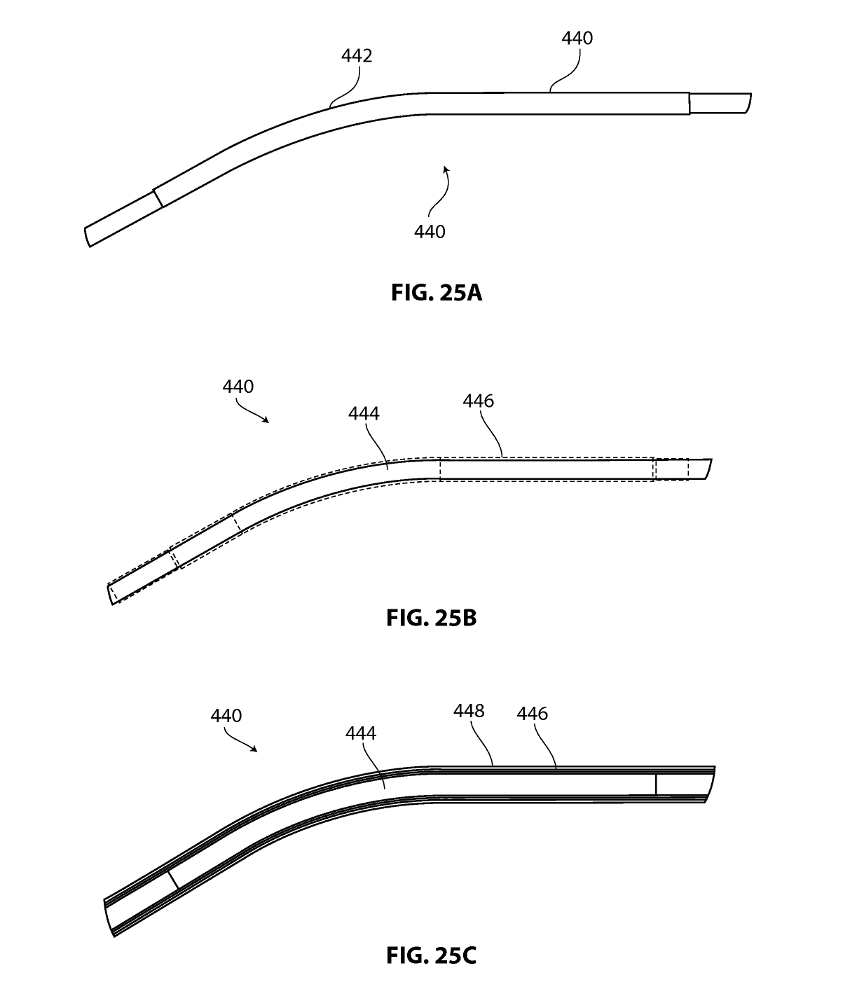

With reference now to FIGS. 25A-25C, in in one embodiment, a tissue cutter/micro-debrider device 440 may include an inner drive shaft 442 that is made, in part, of a flexible multifilar material 444. FIG. 25A shows a portion of the tissue cutter 440, demonstrating that a length of multifilar material 444 may be used to help form the gradual curve in the proximal portion of the inner drive shaft 442. FIG. 25B shows multifilar material 444 covered by a polymer sheath 446, according to one embodiment. In particular, FIG. 25B shows that inner drive shaft 442 includes a proximal portion 442′, a distal portion 442″, and a multifilar portion 444 spanning the gap therebetween and extending over the ends of the proximal portion 442′ and distal portion 442″. As shown, the polymer sheath 446 covers the entire exterior (and/or the interior in some embodiments) of the multifilar material 444, and may extend a predetermined distance beyond the ends of the mulifilar material 444 onto the ends of the proximal portion 442′ and distal portion 442″ of the inner drive shaft 442. FIG. 25C is a cross-sectional view of the tissue cutter 440, showing proximal portion 442′ and distal portion 442″ of the inner drive shaft, multifilar material 444, polymer sheath 446 and an outer tube 448, which resides over sheath 446. This is merely one embodiment of a proximal shaft portion, illustrating one way to form the gradual curve in shaft 442. As mentioned above, in alternative embodiments, any of a number of alternative materials and techniques may be used to form this type of gradual curve or any other desired curve or bend in a proximal shaft portion.

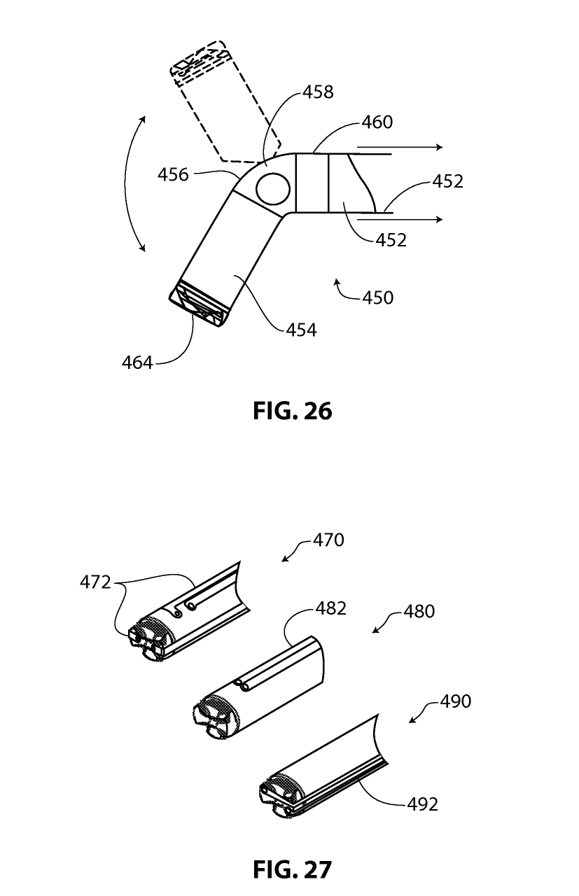

With reference now to FIG. 26, in some embodiments, the sharp, distal bend in a tissue cutter/micro-debrider device may be fixed, in other words with a fixed angle between the proximal shaft portion and the distal shaft portion that is not adjustable by the user. In other embodiments, however, the sharp, distal bend may be adjustable. In some embodiments, the bend may be adjustable by the user manually adjusting the bend by hand before inserting (or reinserting) the device into the patient. In other embodiments, the bend may be adjustable inside the patient, using an adjustment mechanism built into the device. A portion of one such adjustable embodiment is illustrated in FIG. 26. In this embodiment, the tissue cutter/micro-debrider device 450 includes a proximal shaft portion 452, a distal shaft portion 454, a bend 456 between the two portions, a hinge 458 enabling movement at bend 456, two pull wires 460, 462, used to move distal portion 454 about hinge 458, and a cutting member 464 at the distal end of distal potion 454. In one embodiment, as shown, distal portion 454 may move relative to proximal portion 452 to form angles ranging from about 45 degrees to about 135 degrees. In other embodiments, the angles may be ranging from about 90 degrees to about 180 degrees.

Any of the embodiments of the shafts of a tissue cutter/micro-debrider device may be combined with any suitable distal cutting member or other end effector. Various embodiments of these cutting members and end effectors are described in further detail below. In addition to the distal end effectors, in some embodiments, a tissue cutter/micro-debrider may also include one or more energy delivery members for delivering energy to tissue (or removing energy from tissue, in the case of cryotherapy). In some embodiments, for example, radiofrequency (RF) electrodes may be incorporated into the distal portion of the shaft for ablating tissue and/or coagulating blood vessels to reduce bleeding.

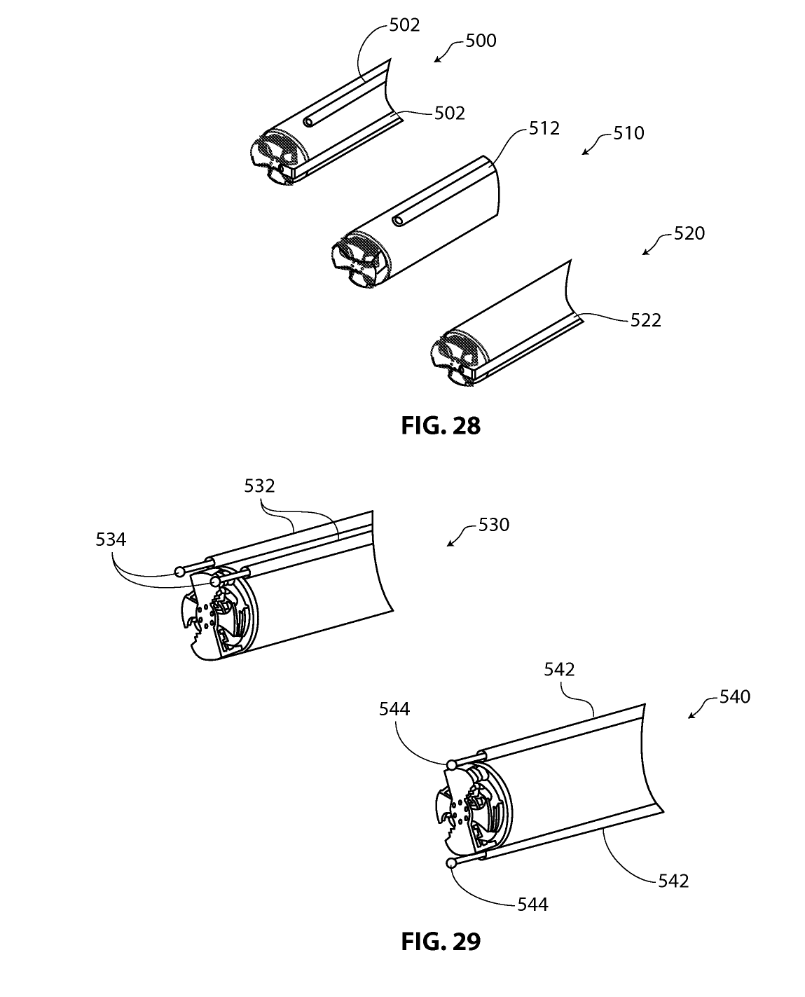

With reference now to FIG. 27, three embodiments of distal ends of tissue cutters 470, 480, 490 are illustrated. In these embodiments, each tissue cutter 470, 480, 490 includes at least two bipolar electrodes 472, 482, 492. As illustrated in the figure, the bipolar electrodes 472, 482, 492 may travel along the length of a distal shaft portion and may be exposed at or near a distal end to provide ablation and/or coagulation. FIG. 28 illustrates alternative embodiments of a tissue cutter 500, 510, 520, in which each embodiment includes at least one monopolar RF electrode.

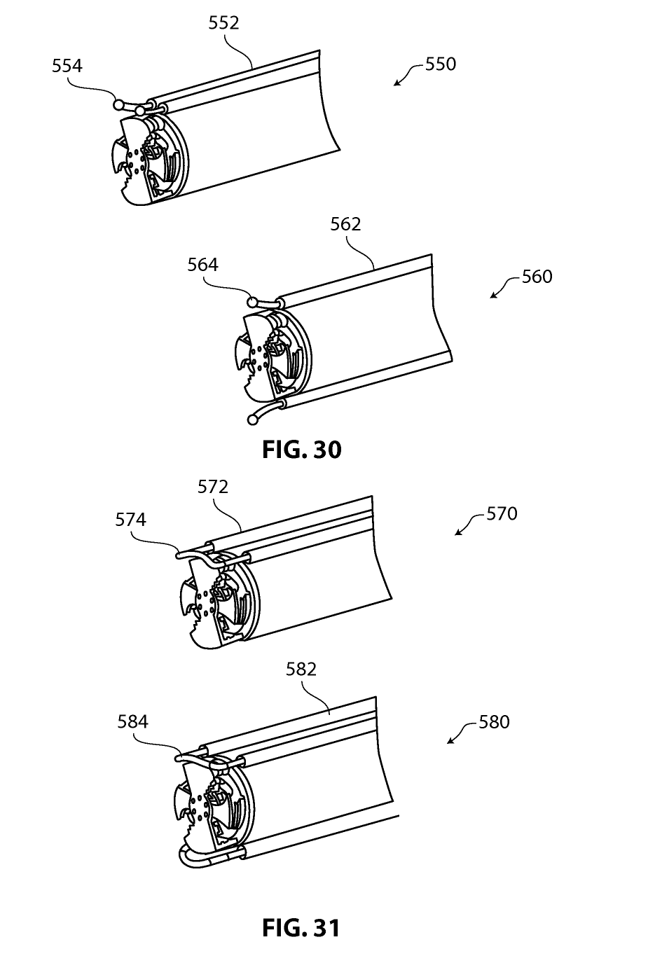

Referring to FIG. 29, in other alternative embodiments, tissue cutters 530, 540 may include one or more sheaths 532, 542, fixed to an external surface of the tissue cutter shaft, to house electrodes 534, 544. FIG. 30 illustrates two additional embodiments of tissue cutters 550, 560 with electrode sheaths 552, 562 and electrodes 554, 564 with curved ends. FIG. 31 illustrates two additional embodiments of tissue cutters 570, 580 with electrode sheaths 572, 582 and looped electrodes 574, 584. The electrodes may be monopolar, bipolar or resistive. In some bipolar embodiments, the electrodes are configured to measure tissue types through impedance. They can also be used to stimulate nerves and evoke a response in the surrounding tissue. From these examples, it becomes apparent that various alternative embodiments of tissue cutter/micro-debrider devices may include any of a number of different configurations of electrodes, electrode sheaths or housings and the like.

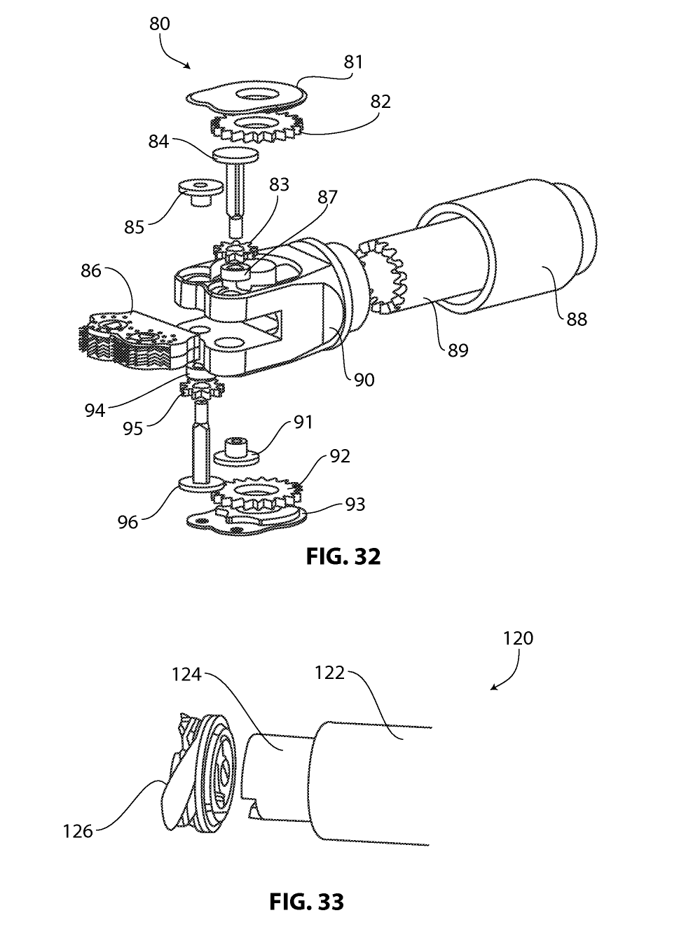

Referring now to FIG. 32, an exploded view of a distal portion of one embodiment of a micro-debrider device 80 is shown. FIG. 32 illustrates the gearing mechanism that drives the blade assembly 86 of device 80. Although each part illustrated in FIG. 32 will not be described in detail, the figure illustrates the parts with sufficient detail to allow one of skill in the art to make and use the gearing. In this embodiment, micro-debrider device 80 includes a housing 88 disposed over a drive tube crown gear 89 (analogous to first gear 67, 77 in FIGS. 18 and 19), which attaches to a lug 90, which holds blade assembly 86. Blade assembly 86 is attached to lug 90 and actuated via two retainers 81, 93, two right angle gears 82, 92, two pins 84, 96, two pin aligners 85, 91, two small gears 83, 95, and two spacers 87, 94. When device 80 is actuated, drive tube crown gear 89 rotates in one direction and then another to drive right angle gears 82, 92 and small gears 83, 95, which in turn drive the blades of blade assembly 86 to rotate in opposite directions relative to one another (i.e., toward one another). As the blades rotate toward one another, they pass very close to one another, thus sheering off tissue (or shredding tissue) between the blades. As described above, the mechanism illustrated in FIG. 32, combined with a middle gear and two additional drive tube crown gears, allows device 80 to have a sharp bend and a small outer diameter.

Referring now to FIG. 33, in another embodiment, a micro-debrider device 120 may include an outer tube 122, inner drive tube 124 and other mechanism as described above. This embodiment, however, includes a concentric cutter 126 rather than the reciprocating blades of the embodiment described above. Concentric cutter 126 generally includes a rotating blade and a stationary blade. The rotating blade rotates in one direction passing in close proximity to the stationary blade and thus cutting tissue between the two blades. In some embodiments, the rotating blade and the stationary blade include multiple blades that interdigitate with each other, such that tissue is shredded between the moving and stationary interdigitating blades.

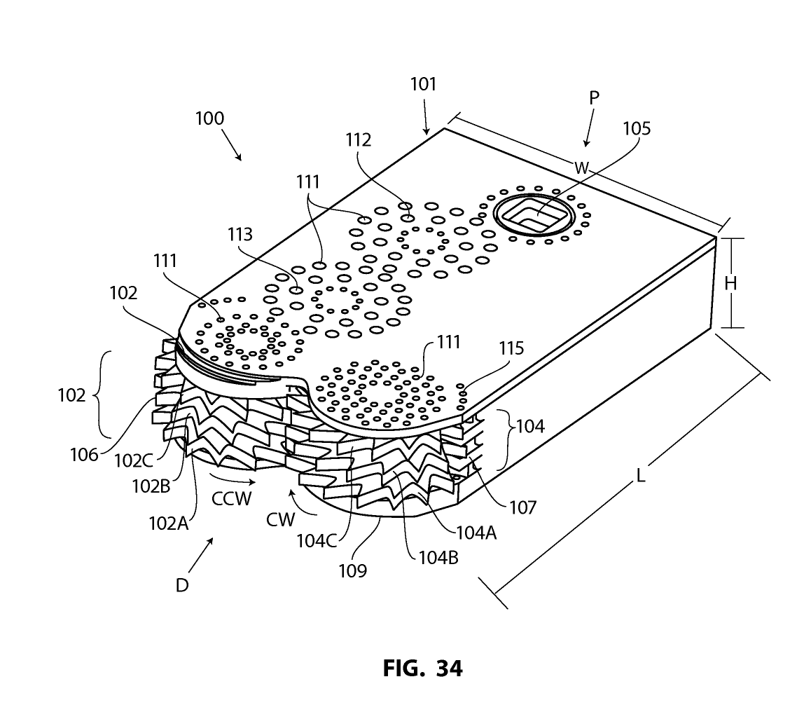

FIG. 34 illustrates an exemplary embodiment of a blade assembly 100 (or “tissue cutter” or “micro-debrider”) of a micro-debrider tissue removal device. Blade assembly 100, which is similar to the embodiment illustrated in lesser detail in FIG. 16, is described in further details and in alternative embodiments in U.S. patent application Ser. No. 13/007,578 (Pub. No. 2012/0109172), which was previously incorporated by reference herein. Tissue removal device working end 100 has a distal region “D” and proximal region “P,” and includes housing 101 and blade stacks 102 and 104. Blade stacks 102 and 104 include a plurality of blades 102A-102C and 104A-104C, respectively. Three blades are shown in each stack, although the blade stacks can have one or more blades. Each of the blades includes a plurality of teeth 106, some of which are shown projecting from housing 101 and configured to engage and process tissue. Processing tissue as used herein includes any of cutting tissue, shredding tissue, capturing tissue, any other manipulation of tissue as described herein, or any combination thereof. The working end of the device generally has a length L, height H, and width W. Housing 101 can have a variety of shapes or configurations, including a generally cylindrical shape.

In this embodiment, both blade stacks are configured to rotate. The blades in blade stack 102 are configured to rotate in a direction opposite that of the blades in blade stack 104, as designated by the counterclockwise “CCW” and clockwise “CW” directions in FIG. 34. The oppositely rotating blades direct material, such as tissue, into an interior region of housing 101 (described in more detail below). In some embodiments, the blades can be made to be rotated in directions opposite to those indicated, e.g. to disengage from tissue if a jam occurs or to cause the device to be pulled distally into a body of tissue when given appropriate back side teeth configurations.

Housing 101 also includes a drive mechanism coupler 105, shown as a square hole or bore, which couples a drive train disposed in the housing to a drive mechanism disposed external to the housing. The drive mechanism, described in more detail below, drives the rotation of the drive train, which drives the rotation of the blades. The drive train disposed in the housing can also be considered part of the drive mechanism when viewed from the perspective of the blades. Drive mechanism coupler 105 translates a rotational force applied to the coupler by the drive mechanism (not shown) to the drive train disposed within housing 101. FIG. 34 also shows release holes 111-115 which allow for removal of sacrificed material during formation of the working end.

Material may be directed into housing 101 by the rotating blades, and housing may include a chamber (not visible) where the cut tissue can be stored temporarily or directed further proximally. In some embodiments in which the working end 100 includes a storage chamber, the chamber may remain open while in other embodiments it may be closed while in still other embodiments it may include a filter that only allows passage of items of a sufficiently small size to exit.

In general, the blades in stack 102 are interdigitated with the blades in stack 104 (i.e. the blade ends are offset vertically along dimension H and have maximum radial extensions that overlap laterally along the width dimension W. The blades can be formed to be interdigitated by, e.g. if formed using a multi-layer, multi-material electrochemical fabrication technique, forming each blade in stack 102 in a different layer than each blade in stack 104. During formation, portions of separately moveable blade components overlap laterally, and in some embodiments the overlapping blades are not just formed on different layers but are formed such that an intermediate layer defines a vertical gap between them. For example, the bottom blade in stack 102 is shown formed in a layer beneath the layer in which the bottom blade in stack 104 is formed.

When manufacturing tissue removal devices of the various embodiments set forth herein using a multi-layer multi-material electrochemical fabrication process, it is generally beneficial, though not necessarily required, to maintain horizontal spacing of component features and widths of component dimensions remain above the minimum feature size. It is important that vertical gaps of appropriate size be formed between separately movable components that overlap in X-Y space (assuming the layers during formation are being stacked along the Z axis) so that they do not inadvertently bond together and to ensure that adequate pathways are provided to allow etching of sacrificial material to occur. For example, it is generally important that gaps exist between a gear element (e.g. a tooth) in a first gear tier and a second gear tier so that the overlapping teeth of adjacent gears do not bond together. It is also generally important to form gaps between components that move relative to one another (e.g., gears and gear covers, between blades and housing, etc.). In some embodiments the gaps formed between moving layers is between about 2 micrometers (um) and about 8 um.

In some embodiments, it is desired to define a shearing thickness as the gap between elements has they move past one another. Such gaps may be defined by layer thickness increments or multiples of such increments or by the intralayer spacing of elements as they move past one another. In some embodiments, shearing thickness of blades passing blades or blades moving past interdigitated fingers, or the like may be optimally set in the range of 2-100 microns or some other amount depending on the viscosity or other parameters of the materials being encountered and what the interaction is to be (e.g. tearing, shredding, transporting, or the like). For example, for shredding or tearing tissue, the gap may be in the range of 2-10 microns, or in some embodiments in the range of 4-6 microns.

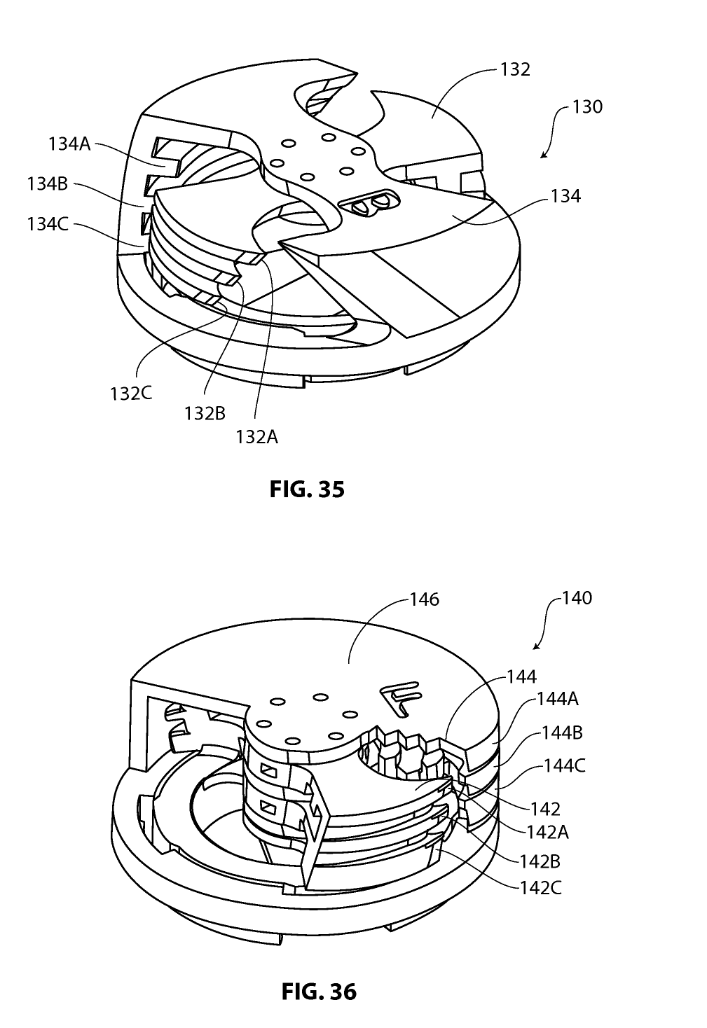

Referring now to FIG. 35, in one alternative embodiment, a blade assembly 130 may include a concentric cutter. In this embodiment, blade assembly 130 includes a rotating (or “concentric”) cutter 132 having multiple blades 132a, 132b, 132c and a stationary cutter 134 having multiple blades 134a, 134b, 134c. Rotating blades 132a, 132b, 132c interdigitate with stationary blades 134a, 134b, 134c so that tissue is cut off between them. As with the previously described embodiments, tissue that is cut or shredded by cutters 132, 134 is typically urged proximally into blade assembly 130 and thus into a chamber and/or conduit of the device. In some embodiments, this proximal movement may be facilitated by suction and/or irrigation.

With reference now to FIG. 36, in another alternative embodiment, a blade assembly 140 may include a concentric cutter. In this embodiment, blade assembly 140 includes a rotating (or “concentric”) cutter 142 having multiple blades 142a, 142b, 142c and a stationary cutter 144 having multiple blades 144a, 144b, 144c. Rotating blades 142a, 142b, 142c interdigitate with stationary blades 144a, 144b, 144c so that tissue is cut off between them. As with the previously described embodiments, tissue that is cut or shredded by cutters 142, 144 is typically urged proximally into blade assembly 140 and thus into a chamber and/or conduit of the device. This embodiment also includes a guard portion 146 on top (i.e., the extreme distal end) of blade assembly 140. Guard portion 146 may help protect nearby tissues from unwanted damage and may thus help facilitate tissue removal procedures near sensitive structures.

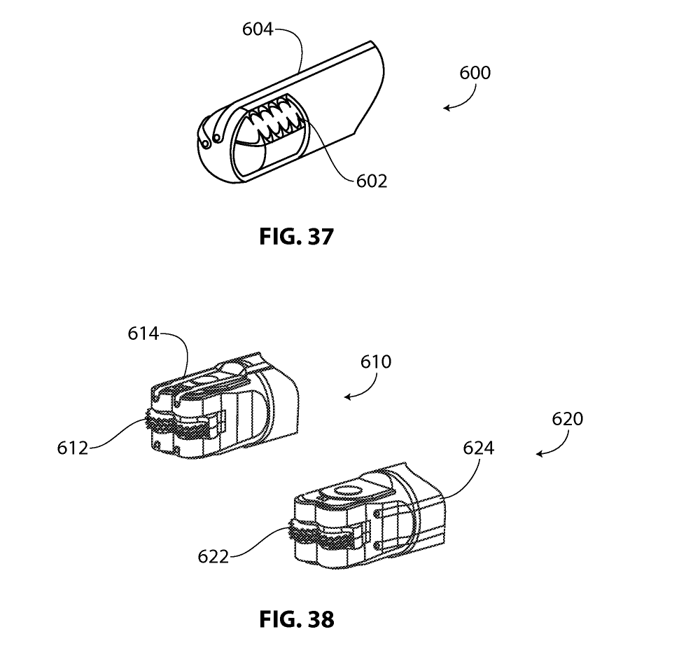

Turning now to FIG. 37, in another alternative embodiment, a tissue cutter/micro-debrider device 600 may include a sideways-facing cutting member 602 and electrodes 604, as described previously. Side-facing cutting member 602 may include multiple, interdigitated teeth, as shown. Electrodes 604 may be monopolar or bipolar.

FIG. 38 illustrates two alternative embodiments of a tissue cutter/micro-debrider device. The first embodiment of tissue cutter 610 includes a forward-facing, tissue shredder cutting member 612 and electrodes 614 located on a forward-facing surface of device 610, above the rotating tissue cutting elements. The second embodiment 620 includes the same type of forward-facing, tissue shredder cutting member 622 with electrodes 624 located on the side of device 620.

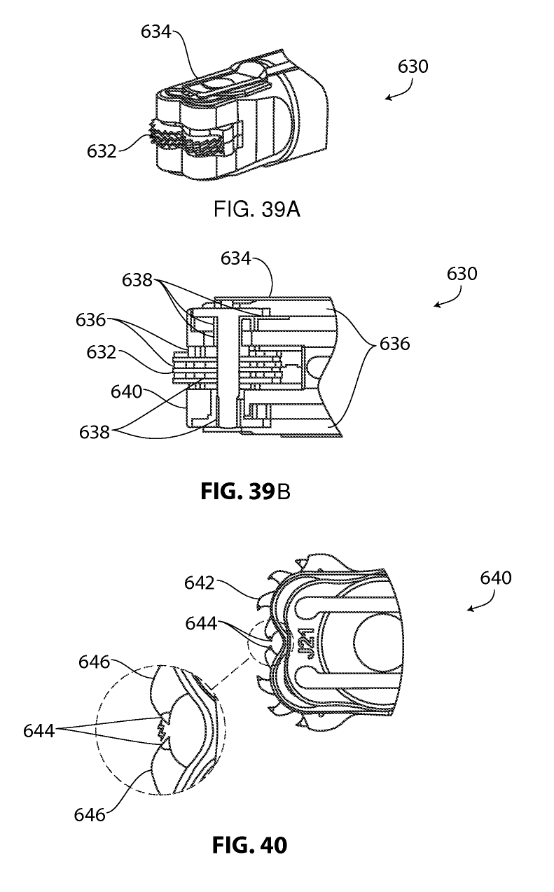

With reference to FIGS. 39A, 39B and 40, three views are provided of another embodiment of a tissue cutter/micro-debrider device 630. In this embodiment, device 630 includes a forward-facing tissue shredder cutting member 632 provided with bipolar conductors 634 located along the top surface. In an alternative version of this embodiment, a first conductor 634 is located along the top surface and a second conductor 634 is located along the bottom surface. Also shown in the cross-sectional view of FIG. 39B are ceramic portions 636, insulation portions 638, and a ceramic coated lug 640, all according to one embodiment of device 630. With this arrangement, conductors 634 provide RF or other energy to a portion of the drive train to ultimately supply the cutting blades (or just the tips of the cutting blades, as described below with reference to FIG. 40) with the energy for enhanced cutting, coagulating, sealing, necrosing, sensing, etc.

Referring now primarily to the cross-sectional view of FIG. 39B, device 630 is constructed and operates in a manner similar to that of previously described device 80 shown in the exploded view of FIG. 32, but has features for delivering energy to the cutting blades 642 and 643, and insulating other portions of the device from receiving that energy. FIG. 39B shows the energy delivery path for one pole of the bipolar energy to one set of the cutting blades 642. The drive train for the other set of oppositely rotating cutting blades 643 can be constructed in a symmetrical fashion such that the other set of cutting blades receives the opposite pole of the bipolar energy. In other embodiments (not shown), monopolar energy can instead be delivered to one or both sets of cutting blades.

Device 630 may be provided with ceramic portions 636 which correspond to the upper and lower retainers 81 and 93 shown in FIG. 32. Conductors 634 may be provided along an outer surface of ceramic portions 636 such that each conductor 634 aligns with the head of one of the pins 84 (as shown) or 96 (not shown). An electrical brush 639 may be provided on a distal, cantilevered section of conductor 634 as shown. The cantilevered section of conductor 634 may be configured to provide a biasing force to urge the electrical brush 639 against the top of pin 84 as it rotates during operation. Electrical (RF) energy is thereby transmitted from conductor 634, through electrical brush 639, through pin 84 to the rotating cutting blades 642 which are attached to pin 84. Electrical energy is inhibited from being transmitted to small gear 83′ and spacer 87′, right angle gear 82′ and aligner 91′ by insulation that may be provided on these components, may be provided on pin 84, or a separate insulation component therebetween. In this embodiment, blade assembly housing 636 is formed of ceramic, and lug 640 is covered with a ceramic coating to further insulate components that are not intended to conduct the electrical energy. Rotating blade hubs 641, which are located between the blades of each of the two blade sets, may also be provided with insulation such that electrical energy is only transmitted between opposing blades rather than between a blade and an opposite blade hub 641.

FIG. 40 illustrates further details of the cutter/micro-debrider device 630. Each tooth (or alternatively only some of the teeth) may include an exposed electrode tip 644, with the rest of the tooth being covered with an insulation material 646. This is illustrated best in the magnified cut-out portion of FIG. 40. As the teeth from oppositely rotating blades 642 and 643 rotate towards each other, the exposed electrode tips 644 may help coagulate blood vessels, cut through tissue or both, in various embodiments.

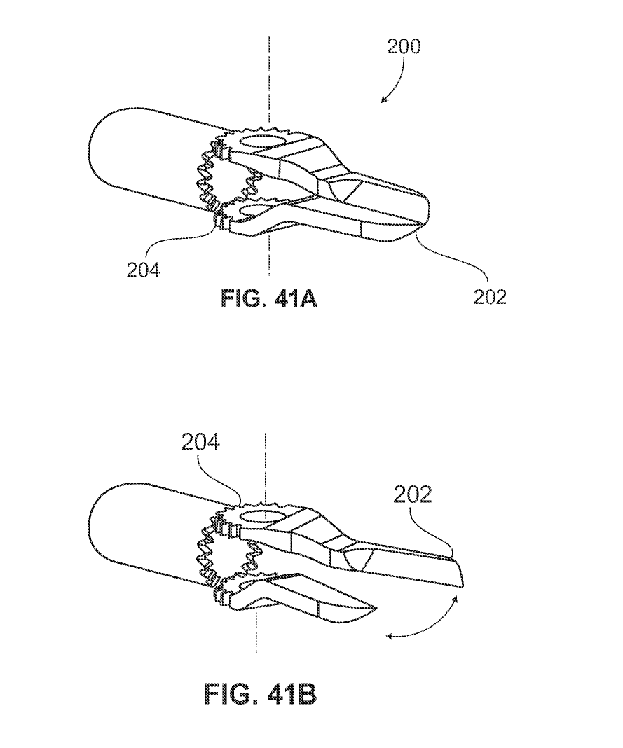

Referring now to FIGS. 41A and 41B, in one embodiment, a tissue removal or manipulation device may include, rather than tissue cutters like those described above, an opposable end effector tool 200. In this embodiment, end effector tool 200 includes opposable micro-shears 202 (or “scissors”), which are driven to open and close by a crown gear 204. Like the above-described tissue cutters, micro-shears 202 may be so small that they may be used to cut and/or remove extremely small amounts of tissue at each cut, thus helping prevent unwanted tissue damage.

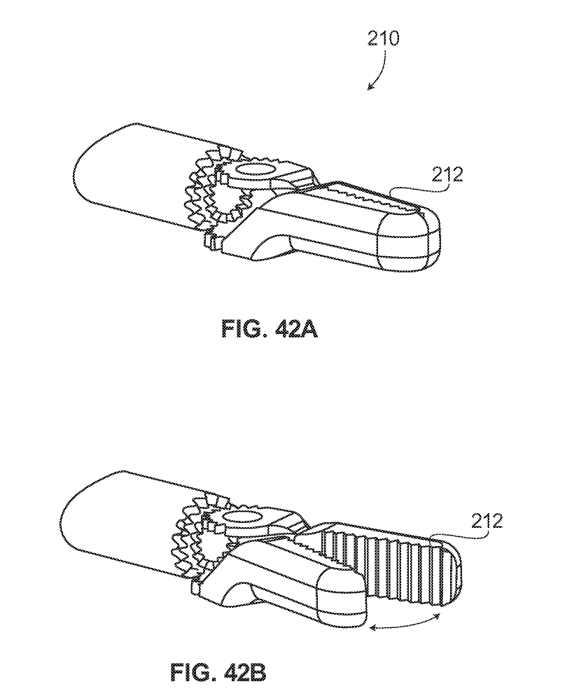

Referring to FIGS. 42A and 42B, another embodiment of an end effector 210 may include graspers 212. In one embodiment, graspers 212 may be coupled with an RF energy source to provide bipolar tissue ablation and/or cautery along with grasping force.

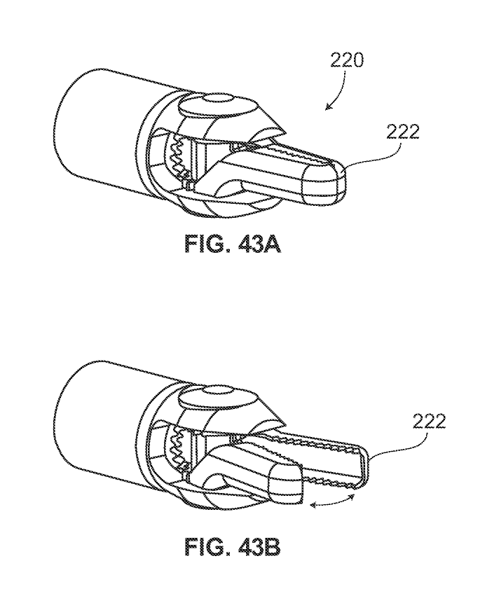

Referring to FIGS. 43A and 43B, in another embodiment, an end effector 220 may include biopsy forceps 222. Again, in one embodiment, forceps 222 may also be bipolar RF energy forceps.

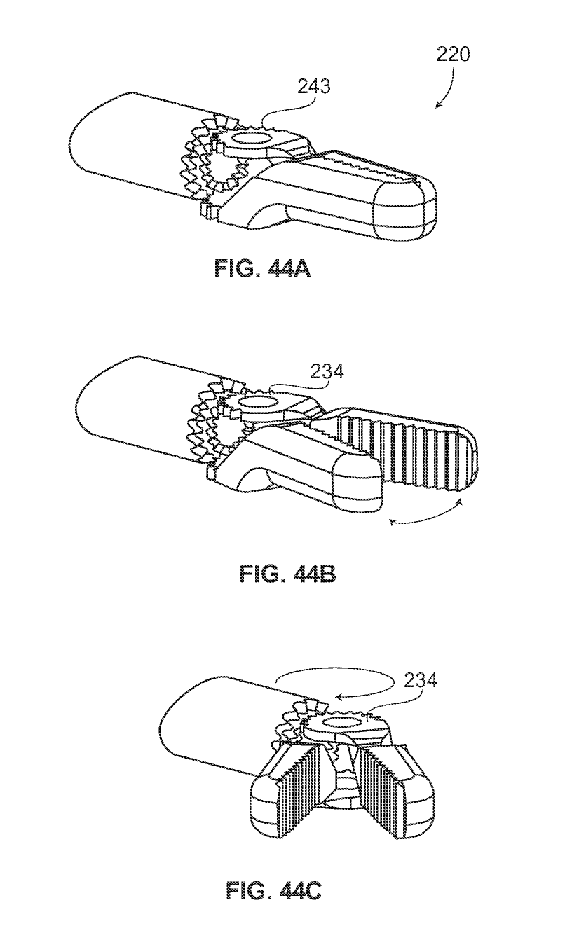

Referring to FIGS. 44A-44C, in one embodiment, an end effector 230 may include an articulating wrist 234, which allows end effector 230 to articulate about an axis, as shown in FIG. 44C. In some embodiments, articulation may be accomplished via a crown gear. In the embodiment shown, for example, end effector 230 includes at least two crown gears—one for opening and closing one side of the graspers and one for opening and closing the other side of the graspers. When the two sides of the graspers are rotated in opposite directions, the graspers open or close. When the two sides of the graspers are rotated in the same direction, they are articulated around wrist 234. In other embodiments (not shown), one crown gear can be used to open and close both sides of the graspers while a second crown gear can be used to articulate both sides of the graspers.

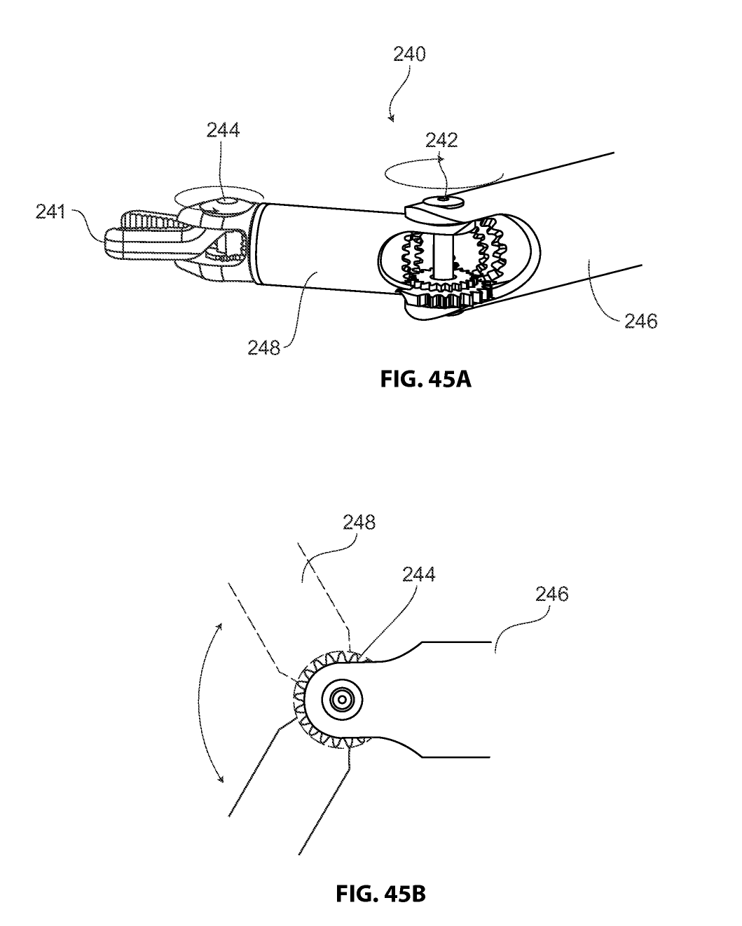

Referring to FIGS. 45A and 45B, in yet another alternative embodiment, a micro-debrider device 240 may include a proximal wrist 242 and a distal wrist 244. Proximal wrist 242 connects a proximal shaft portion 246 (or “second arm”) to a distal shaft portion 248 (or “first arm”). Distal wrist 244 connects distal shaft portion 248 with an end effector 241, which in this embodiment is a pair of graspers. FIG. 45B illustrates the variation in angles that this embodiment may be moved to at the proximal wrist 242.

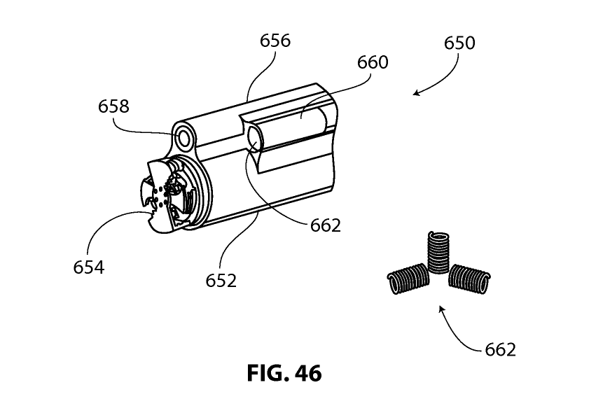

Referring now to FIG. 46, in yet another embodiment, a tissue cutter/micro-debrider device 650 may include a shaft 652, a distal-end, forward-facing cutting member 654, a first sheath 656 on the outside of shaft 652 for holding a camera 658 or other visualization device, and a second sheath 660 on the outside of shaft 652 for holding navigation coils 662 used as part of an image guidance/navigation system. Camera 658 may be any suitable visualization device, such as but not limited to a CCD camera, a CMOS camera, a fiber optic scope, or the like. Camera 658 may also include illumination in some embodiments. Coils 662 may be used to generate a low intensity magnetic field, for implementing an image guidance system. In alternative embodiment, second sheath 660 may be eliminated, and only camera 658 may be included. In other embodiments, additional sheaths may be added and additional features included in the added sheaths.

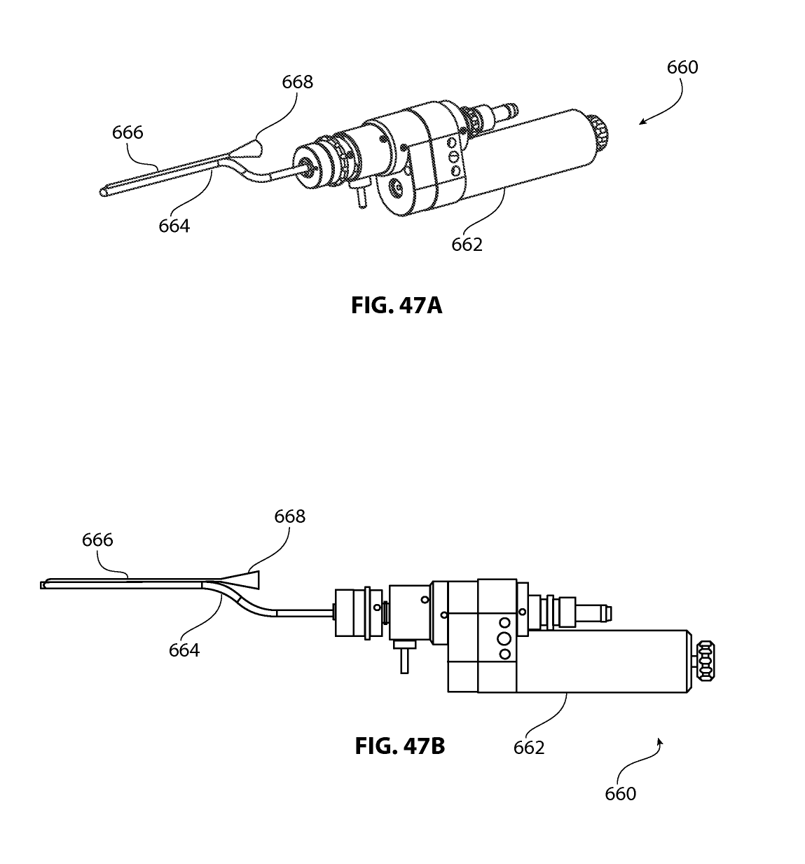

With reference now to FIGS. 47A and 47B, in another alternative embodiment, a tissue cutter/micro-debrider device 660 may include a handle 662 and a shaft 664 (both as previously described in detail above), as well as a guide member 666 attached to the outside surface of shaft 664. Guide member 666 may have a tubular, ovoid or other convenient cross-sectional shape and may include a proximal funnel 668, for helping guide one or more instruments through guide member 666. Guide member 666 may be used, for example, to help guide a flexible guidewire, needle, endoscope, ablation device or other instrument(s) alongside shaft 664. Guide member 666 may be made of metal, polymer or other material and may be attached to shaft 664 by any suitable adhesive or other means.

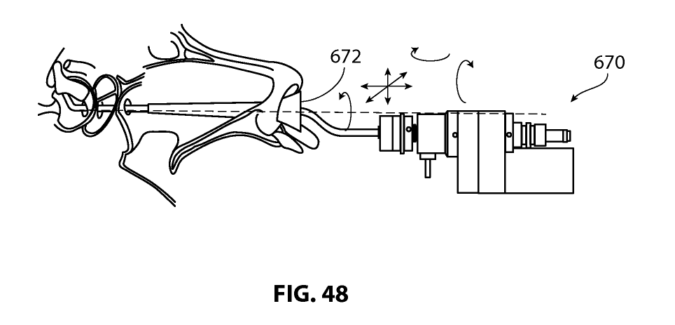

In some embodiments, and with reference now to FIG. 48, a tissue cutter/micro-debrider device 670 may be configured for use with a robotic surgery system. In one such embodiment, tissue cutter 670 may include a speculum 672, which may be attached to, or alternatively separate from, the shaft of tissue cutter 670. Speculum 672 acts as a reference device for using and manipulating tissue cutter 670. In an alternative embodiment, speculum 672 may be used with tissue cutter 670 in a manual, non-robotic method.

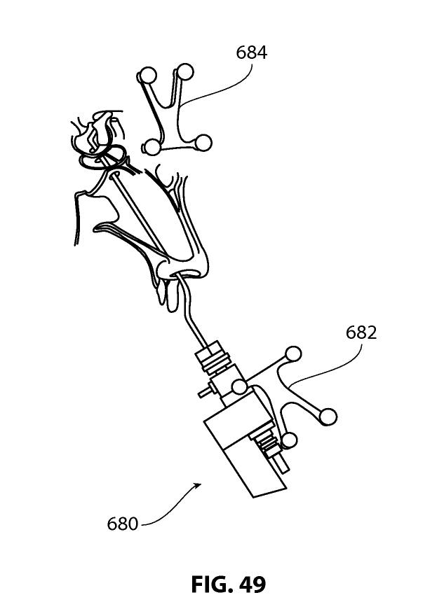

Referring to FIG. 49, in some embodiments, a tissue cutter/micro-debrider device 680 may also be used with any of a number of image guidance systems. In one embodiment, for example, tissue cutter 680 may be used with an infrared image guidance system, which may include a first image guidance reflector 682, attached to tissue cutter 680, and a second image guidance reflector 684, attached to the patient. In various embodiments, any suitable image guidance system may be used.

Elements or components shown with any embodiment herein are exemplary for the specific embodiment and may be used on or in combination with other embodiments disclosed herein. The invention is susceptible to various modifications and alternative forms and should not be limited to the particular forms or methods disclosed. To the contrary, the invention is to cover all modifications, equivalents and alternatives thereof.

What is claimed is:

1. A method for removing at least part of a pituitary tumor in a patient, the method comprising:
advancing a distal end of a tissue cutter through a nostril and through the sphenoid sinus of the patient to contact a cutting member of the tissue cutter with the pituitary tumor, wherein the tissue cutter includes an outer shaft configured to enter the nostril and having an outer diameter no greater than about 10 mm, which includes a distal shaft portion and a proximal shaft portion, and wherein the distal shaft portion is sharply angled relative to the proximal shaft portion;
activating the cutting member to cut tissue from the pituitary tumor by rotating an inner drive shaft located within the outer shaft; and
moving the cut pituitary tumor tissue through a channel within at least one of the shafts toward a proximal end of the tissue cutter.

2. A method as in claim 1, wherein the cutting member does not extend laterally beyond the outer diameter of the tissue cutter outer shaft.

3. A method as in claim 1, further comprising, before contacting the pituitary tumor:
forming an opening through the sphenoid sinus; and
advancing the distal end of the tissue cutter through the opening.

4. A method as in claim 3, wherein the opening is formed using the tissue cutter.

5. A method as in claim 1, wherein cutting the tissue comprises shredding the tissue.

6. A method as in claim 1, wherein moving the tissue comprises urging the tissue into the channel with a cutting motion of the tissue cutter.

7. A method as in claim 6, wherein moving the cut tissue through the channel further comprises applying suction to the channel.

8. A method as in claim 7, wherein moving the cut tissue through the channel further comprises introducing fluid, via the tissue cutter, to an area at or near the distal end of the tissue cutter, wherein the applied suction moves at least some of the fluid proximally through the channel with the cut tissue.

9. A method as in claim 1, wherein the cutting member comprises at least one moveable blade and at least one stationary blade, and wherein cutting tissue comprises rotating the at least one rotating blade past the at least one stationary blade.

10. A method as in claim 1, wherein the cutting member comprises at least two interdigitated blades, and wherein cutting tissue comprises rotating the two interdigitated blades toward one another to shear tissue therebetween.

11. A method as in claim 1, wherein the cutting member is selected from the group consisting of micro-shears, graspers and biopsy forceps.

12. A method as in claim 1, wherein the distal shaft portion is angled relative to the proximal shaft portion by at least 1 degree.

13. A method as in claim 1, wherein the distal shaft portion is angled relative to the proximal shaft portion by at least 45 degrees.

14. A method as in claim 1, wherein the distal shaft portion is angled relative to the proximal shaft portion by about 90 degrees.

15. A method as in claim 12, wherein the proximal shaft portion is curved.

16. A method as in claim 1, further comprising visualizing the tissue cutting using a visualization device selected from the group consisting of a straight endoscope, an angled endoscope, a swing prism endoscope, a side viewing endoscope, a flexible endoscope, a CMOS digital camera, an ultrasound device and a scanning single fiber endoscope.

17. A method as in claim 14, wherein the visualization device is incorporated into the tissue removal device.

18. A method as in claim 1, further comprising measuring an amount of the removed tissue by filtering the removed tissue from a stream of irrigation fluid.

19. A method as in claim 1, further comprising measuring an amount of the removed tissue by determining motor torque in the tissue removal device during engagement of the device with the tissue and using at least one of the determined motor torque, a time period of tissue removal or a loading condition to approximate the amount of the removed tissue

20. A method as in claim 1, further comprising monitoring a location of the tissue removal device during use, using a navigation system and at least one tracking feature on the device.

21. A method as in claim 1, further comprising collecting a sample of cut tissue, using a tissue capturing feature on the device, for use as a histological sample.

22. A method as in claim 1, further comprising at least partially removing a blood clot from the patient through the channel, wherein removing the blood clot includes breaking up the clot using the cutting member.

23. A method as in claim 1, wherein the tissue cutter is coupled with an image guided or robotic surgical system during performance of at least part of the method.

24. A method as in claim 1, further comprising protecting tissues not intended for treatment from contacting the cutting member during use of the device.

25. A method as in claim 1, further comprising:
stimulating a portion of the pituitary tumor using a stimulation member at or near the distal end of the tissue removal device; and
deciding whether to cut the stimulated tissue, based on an observed response from the stimulation.

26. A device for removing at least part of a pituitary tumor, the device comprising:
an outer shaft comprising a distal end, a proximal end, a distal shaft portion, a proximal shaft portion, a sharp bend at a juncture of the distal shaft portion and the proximal shaft portion, a channel extending from the distal end through at least part of the proximal portion, and an outer diameter no greater than about 10 mm;
at least one moveable cutting member disposed at the distal end of the shaft such that, in use, the cutting member does not extend laterally beyond the outer diameter of the outer shaft;
a handle coupled with the proximal portion of the outer shaft;
an actuator coupled with the handle and the at least one cutting member to allow a user to activate the at least one cutting member via the handle, the actuator comprising an inner drive shaft configured to rotate about a central longitudinal axis when activating the at least one cutting member; and
at least one aperture on at least one of the handle or the proximal shaft portion and in fluid communication with the channel, for providing at least one of attachment to a source of suction force or withdrawal of cut tissue through the aperture.

27. A device as in claim 26, wherein the distal portion has a length of no more than about 25 mm, and wherein the bend forms an angle between the distal shaft portion and the proximal shaft portion of at least about 5 degrees.

28. A device as in claim 26, wherein the channel extends from the distal end of the outer shaft to the at least one aperture.

29. A device as in claim 26, further comprising a suction port on the proximal portion or the handle for applying suction to the channel.

30. A device as in claim 29, further comprising an irrigation port on the proximal portion or the handle for applying irrigation fluid to the channel.

31. A device as in claim 30, wherein the suction port is in fluid communication with the channel which serves as a suction channel in the inner drive shaft of the device, and wherein the irrigation port is in fluid communication with an irrigation channel comprising a space between an outer surface of the inner tube and an inner surface of the outer shaft of the device.

32. A device as in claim 26, wherein the at least one moveable cutting member comprises:
at least one rotating blade; and
at least one stationary blade positioned relative to the rotating blade such that tissue is cut between the rotating blade and the stationary blade.

33. A device as in claim 26, wherein the at least one moveable cutting member comprises multiple interdigitated blades that rotate toward one another to shred tissue.

34. A device as in claim 26, wherein the at least one moveable cutting member is selected from the group consisting of micro-shears, graspers and biopsy forceps.

35. A device as in claim 26, further comprising at least one tubular crown gear for driving the at least one cutting member.

36. A device as in claim 35, wherein the at least one tubular crown gear comprises two tubular crown gears coupled together with at least one intermediate gear disposed between them.

37. A device as in claim 36, wherein the intermediate gear is disposed at the bend in the outer shaft.

38. A device as in claim 26, further comprising an energy transmission member coupled with the distal tip of the outer shaft for transmitting energy to the pituitary tumor, wherein the energy transmitted by the energy transmission member is selected from the group consisting of radiofrequency, ultrasound, microwave, heat and laser energy.

39. A device as in claim 26, further comprising a visualization lumen coupled with an outer surface of the outer shaft, for holding at least a portion of an elongate visualization device.

40. A device as in claim 26, wherein the proximal portion of the outer shaft is curved.

41. A device as in claim 26, further comprising at least one attachment member for attaching the device to an image guide or robotic surgical system.

42. A device as in claim 26, wherein the distal shaft portion includes a safety portion that extends along one side of the cutting member to prevent tissues not intended for treatment from contacting the cutting member during use of the device.
